SEQUENCE LISTING (14 sequences)

SEQ ID NO: 1 - DNA, 3611 bp, Homo sapiens
  misc_feature (0)...(0): cDNA AEG-1
tcctggcggcggcggagtgaggctgacagcggggaacctgggagacccctccgccctccccgcggtggcagcggccgatccccggctccggcgcgagggacggccgcgatgcgctcggcctgaggttacccggcccggcccttcctcgcttccctcgactattccactgcgtctccgcgccccggcgtcatcctgcgagtccctctgacgggagggaagatggctgcacggagctggcaggacgagctggcccagcaggccgaggagggctcggcccggctgcgggaaatgctctcggtcggcctaggctttctgcgcaccgagctgggcctcgacctggggctggagccgaaacggtaccccggctgggtgatcctggtgggcactggcgcgctcgggctgctgctgctgtttctgctgggctacggctgggccgcggcttgcgccggcgcccgcaaaaagcggaggagcccgccccgcaagcgggaggaggcggcggccgtgccggccgcggcccccgacgacctggccttgctgaagaatctccggagcgaggaacagaagaagaagaaccggaagaaactgtccgagaagcccaaaccaaatgggcggactgttgaagtggctgagggtgaagctgttcgaacacctcaaagtgtaacagcaaagcagccaccagagattgacaagaaaaatgaaaagtcaaagaaaaataagaagaaatcaaagtcagatgctaaagcagtgcaaaacagttcacgccatgatggaaaggaagttgatgaaggagcctgggaaactaaaattagtcacagagagaaacgacagcagcgtaaacgtgataaggtgctgactgattctggttcattggattcaactatccctgggatagaaaataccatcacagttaccaccgagcaacttacaaccgcatcatttcctgttggttccaagaagaataaaggtgattctcatctaaatgttcaagttagcaactttaaatctggaaaaggagattctacacttcaggtttcttcaggattgaatgaaaacctcactgtcaatggaggaggctggaatgaaaagtctgtaaaactctcctcacagatcagtgcaggtgaggagaagtggaactccgtttcacctgcttctgcaggaaagaggaaaactgagccatctgcctggagtcaagacactggagatgctaatacaaatggaaaagactggggaaggagttggagtgaccgttcaatattttctggcattgggtctactgctgagccagtttctcagtctaccacttctgattatcagtgggatgttagccgtaatcaaccctatatcgatgatgaatggtctgggttaaatggtctgtcttctgctgatcccaactctgattggaatgcaccagcagaagagtggggcaattgggtagacgaagaaagagcttcacttctaaagtcccaggaaccaattcctgatgatcaaaaggtctcagatgatgataaagaaaagggagagggagctcttccaactgggaaatccaaaaagaaaaaaaagaaaaagaagaagcaaggtgaagataactctactgcacaggacacagaagaattagaaaaagagattagagaagaccttccagtgaatacctctaaaacccgtccaaaacaggaaaaagctttttccttgaagaccataagcactagtgatccagccgaagtactcgtcaaaaatagccagcctatcaagactcttccacctgctacttctaccgagccatctgtaatcttatcaaaaagtgattctgacaagagctcttcccaagtgccgccaatactacaagagacagataaatccaagtcaaataccaagcaaaatagtgtgcctccttcacagaccaagtctgaaactagctgggaatctcccaaacaaataaaaaagaagaaaaaagccagacgagaaacgtgaaattttttttcctgaattggacatgtgtttgcaaacacttgtcttgaagattatgctgtttatgcaataatttgtgaacatgtacagagttttatataaatttaaaccaatttttaaaacaaaactgcggacaccaccataaaaatggaatcaaaagaaagttaatttatgaaattaagaggtcagcagaatatactcagtgatggaagacacttgggaaagtctttttaatagaacaagaacgatcttaatttaagaatattatcctggtttaacaacagtgccctgtttacaacagattgtgccctatctcatctgcagccgaggaataaaggattctgattagaaagagggttgcctacagattagtaagcaattccttggatcttatgcacagaacttgtaccatttgaatctgttttatgcttaaatcaaagtgctttgatcaaatgcataacctgccatatctttacatatttgttggtagcaatttgtattaaagaaatcacaagtgcaaataaaaagtcatttatcatttgtttaactaaactgtcatggtttagtttacaatttttaaaaagttcttaaaatactgaaaatgcagttgacacttgtgtatggcttatgaagttatttttgatagtcttacattacttgaattgttcaaagtacagtatattttaaattaagaaaagtgaactatatgtatttgttttatacatttaaggcttagactcataaataatgctattgtttatgatttgaaaactttcaggcaaaatccaatttacatttttcccttccctagcaattacttttttccagcttcaactcttcttagttactaatactttgttgactttaaaaatgaaatcattcacaaacttttggtatatgatggagaatgaaaaactagagtcagacagctttaattgacattgtcaacacctccagttatcaggaatacatttttttactgccttaacctgtagtgcgtagaatatgcatcaatttcttgaaggagattcatgtttttataagaattttcatgtaattattgcaattgtggtcaaataaggaacgtttcctgcttgaaattatattgatttaaatgatgtgtgagatgtttcaccattttcaggcactgtgtaattctattgtaataaactggcaggtatctttgtaactataaatagtgcatgctcagccatgtacactgtaaatagcctttaccaaacgtgtttgacaaggaccataattaacatcacttagtgaattgtgataaagaaaaaaaagccatgatttattcgatgtgattggcttgtttttatgtggcgccaagaacgaacctgtttaacagctgtaaccaatggtactgatctatccatccaatgttgtcattatatttgactgtggttcaacagtattgcgttgtcagactaggaaagctaaacgaacaaaatggttttagttttgctgaagactggccttattaatggacagctttcctaacaagagattattaacttttatcaggtgttaacatctgtttcaggaacatggcagtatgtttacatgtcagaagttttgtttaattctatggtatttctaaattgacttgtttaaataaattcagcaaatgg

SEQ ID NO: 2 - DNA, 2759 bp, Homo sapiens
  misc_feature (0)...(0): AEG-1 promoter
ggtaccctttagtaatccctccctctctcccatttctccttcctcccaccaccctccacccccatccccaaggaatcactgatttgctttgtcactatagattagtttgcattttcaagttttatgtaaatggaatcatacaccatgtactcttttttatctggcttctttcactcagcataatccatgttgttacatgtattcatagttcattcctttttatttctgggtcgtattccactgtatgggtcactcccagaaaaagcagtccttatttccaacccggtgcagagctgcaggtaaacggtttcatacacaacagcttaactttgtcatttacaagaaattagaattcccactttaggccagacttgattttaacttctttacatatggcaccacttcaatttaaaggtttttttgttttgttttgagacagaatctccttctgtcacccaggctggagtgcagtagcacgatctcagctcactgcagcctccaccttctgggttcaagttattttcatgcctcagccacctgagtagctgggattacaggcgtgagccacagcaccagccattattgtaatagtttaatttgaaaattcacaggtgggccgggagcggtggctcacgcctgtaatcccagcactttgggaggcccatgcgcgcggatcacgaagtcagggcgactgcagcctcggcaacacagcgagactccatctcaaaaaataaaaaataaaaaattcacagatgtttatctagaatggtcagaatagtcagtggaacttcttaggaaaatcatgacccttcacatctctagaaccaaaacacgtgcagaaatcttatttcacaaatataaatggaatttggaaactttgagttaaagcagatcccaataacatttgctaagtttttggcctcctaatttaatttagtctttaatcataagtcttggcaagtgtagtatgcttttattatttattatttacaaaactcccttccacacatgaatcacaatatgcattacgacactgttgcaaaataggtcaaaatacatttttggtatttggtggttattaatattacccctgctgtattccttgagaaagcctgtatttagttcacttaagacagttgcttacacgctaatgatcatttaccgataaaatacagagtttccttatagcaaaatataatcttaaaaccctaaggcagccttcaagcatttcccttataattaaaacctgaaggcctggcgtggtggctcacgcctgtaatcccagcactttgggaggctgaggtgggcggatcacgaggtcaggagatcaagaccatcctggctaacacggtgaaaccccgtctctactaaaaatacaaaaaattagccgggcgtggtggtgggtgcctatagtcccagctgctcgggaggctgaggcaggagaatggcttgaacccaggaggcggaccttgcagtgagcggagatagtgccactgcactccagcccgggcgacagagcgagactcttgtctcaaaaaaaaaaaatttgaaaaggcagttcgtttccttctatagctgcatcccagagatggtcaacatagcatataaaaatggaatttttgcaaaccccttttgcagtgttgtagaaaagcttaaaataaaacgtaacgagaaagctccctccataagtaaatacaatctaatgtgcttccccttatgtgttattaaagctacaaattagtaggaaaagaatagtgccgcccacgggctttccaacttttgaattctggggactaacaacagagagcaagagtgaatgagccaaacgacgcaaacgtgccctcgccaggctggcaactggtaggcacgcagtgtattagttgccctggagagaaacaccctggagagaaatacccaatttgtgaactaaacctaggtcccggaagaacagtgttcggcgtcagactccgttgagctcattctggaaggatccaactggcgccaccagggagaaaaagcgattccacctcaataacactccagaaaaaggcatgaagagccctatacctgccagggcgactttgacctagacccggtgacccggttcctagcgctgcagccctacccgccccccgcccgcccccgccttgcacggagcccctcctctgtactcattcgttgcgccacgtctcctaactctgcgccaccagccaccccgcgaaggcgtccaccaattaacccctcccagcttctggtctacagtaacgggtccccaacgccgcgtcttaaccaggcccgaaccgaccaccgccaaccttccctgacacgcctttgccccacccgaccccgcccgcccccacgtgacgcccacgccccgcccactggcgcccacgtgacccacggtcgtccccgcgcgcggcgtggatcgcggcccaagccgccattgttccgccgagggaggacagcggggcctggcgctggcgccgagacgccgcttagcggccgccactggagacactccctcccgcctcccgggtctcctggcggcggcggagtgaggctgacagcggggaacctgggagacccctccgccctccccgcggtggcagcggccgatccccggctccggcgcgagggacggccgcgatgcgctcggcctgaggttacccggcccggcccttcctcgcttccctcgactattccactgcgtctccgcgccccggcgtcatcctgcgagtccctctgacgggagggaagat

SEQ ID NO: 3 - DNA, 19 bp, Artificial Sequence
  siRNA for the AEG-1 gene corresponds to the 449-467 nucleotides of the AEG-1 Open reading frame
agcagccaccagagattga

SEQ ID NO: 4 - DNA, 28 bp, Artificial Sequence
  Synthetic oligonucleotide Forward PCR primer for full length promoter
ggtaccctttagtaatccctccctctct

SEQ ID NO: 5 - DNA, 30 bp, Artificial Sequence
  Reverse PCR primer for full length promoter
ctcgagatcttccctcccgtcagagggact

SEQ ID NO: 6 - DNA, 55 bp, Artificial Sequence
  oligo dT adaptor primer , reverse
ttctggtcgactagtgtttaaactcgagactttttttttttttttttttttttvn

SEQ ID NO: 7 - DNA, 26 bp, Artificial Sequence
  anchor primer/GSP3
ctgcctggagtcaagacactggagat

SEQ ID NO: 8 - DNA, 34 bp, Artificial Sequence
  C-terminal hemagglutinin (HA)-tagged AEG-1 forward PCR primer
cgggatccatggctgcacggagctggcaggacga

SEQ ID NO: 9 - DNA, 50 bp, Artificial Sequence
  C-terminal hemagglutinin (HA)-tagged AEG-1 reverse PCR primer
cgggatccctcgagtcacagcgaagcgtagtctgggacgtcgtatgggta

SEQ ID NO: 10 - DNA, 21 bp, Artificial Sequence
  Scrambled control siRNA sequence
gggtcgtctatagggatcgat

SEQ ID NO: 11 - DNA, 40 bp, Artificial Sequence
  Forward PCR primer for AEG-1 cDNA cloning into Baculovirus expression vector
tcccctatactaggtaaaatcgaagaaggtaaactggtaa

SEQ ID NO: 12 - DNA, 26 bp, Artificial Sequence
  Reverse PCR primer for AEG-1 cDNA cloning into Baculovirus expression vector
cttcctttcgggctttgttagcagcc

SEQ ID NO: 13 - DNA, 26 bp, Artificial Sequence
  Forward PCR primer for AEG-1 promoter deletion with boundary at position 1146
ggtaccgaatttttgcaaaccccttt

SEQ ID NO: 14 - DNA, 30 bp, Artificial Sequence; identical to SEQ ID NO: 5
  Reverse PCR primer for AEG-1 promoter deletion with boundary at position 1146

(19) United States
(12) Patent Application Publication
(10) Pub. No.: US 2008/0200412 A1
(43) Pub. Date: Aug. 21, 2008

(54) Astrocyte Elevated Gene-1 And Its Promoter In Treatments For Neurotoxicity And Malignancy

(76) Inventor: Paul B. Fisher, Richmond, VA (US)

Correspondence Address: BAKER BOTTS L.L.P., 30 ROCKEFELLER PLAZA, 44TH FLOOR, NEW YORK, NY, 10112-4498, US

(21) Appl. No.: 11/781,512

(22) Filed: Jul. 23, 2007

Related U.S. Application Data
(63) Continuation of application No. PCT/US05/06639, filed on Feb. 25, 2005.

(51) Int. Cl. A61K 31/70; C12N 15/00; C07H 21/04

(52) U.S. Cl. 514/44; 435/455; 536/24.5; 435/320.1

(57) ABSTRACT

The present invention is based, at least in part, on the discovery that Astrocyte Elevated Gene-1 (‘AEG-1’) expression (i) suppresses the Excitatory Amino Acid Transporter-2 (‘EAAT-2’) promoter, thereby inhibiting glutamate transport; (ii) supports anchorage independent colony formation of cells, in which it is synergistic with the RAS oncogene; and (iii) is increased in a number of different malignancies. The invention, in various embodiments, provides for methods of treatment of malignancies and neurodegenerative disorders using inhibitors of AEG-1 activity, and provides for screening assays for identifying other compounds that have therapeutic benefit.

### STATEMENT REGARDING FEDERALLY SPONSORED RESEARCH

The subject matter of this application was developed, at least in part, under National Institutes of Health grants NS31492, GM068448, so that the United States Government has certain rights herein.

### 1. INTRODUCTION

The present invention is based, at least in part, on the discovery that Astrocyte Elevated Gene-1 (“AEG-1”) expression (i) suppresses the Excitatory Amino Acid Transporter-2 (“EAAT-2”) promoter, thereby inhibiting glutamate transport; (ii) supports anchorage independent colony formation of cells, in which it is synergistic with the RAS oncogene; and (iii) is increased in a number of different malignancies. It is also based, in part, on the discoveries of the AEG-1 promoter and its increased activity in the presence of activated mutant RAS. The invention, in various embodiments, provides for methods of treatment of malignancies and neurodegenerative disorders using inhibitors of AEG-1 activity or gene therapy constructs in which the AEG-1 promoter drives expression of a therapeutic gene, and provides for screening assays for identifying other compounds that have therapeutic benefit.

### 2. BACKGROUND OF THE INVENTION

HIV-1 infection of astrocytes contributes to Human Immunodeficiency Virus (HIV) Associated Dementia (“HAD”) by diverse mechanisms, including astrogliosis and glutamate excitotoxicity (7). Astrogliosis resulting from HIV infection involves the activation of astrocyte proliferation followed by apoptosis (8). Perturbation of astroglial-neuronal interactions and secretion of neurotoxic cytokines and chemokines in astrogliosis can also contribute to neuronal cell atrophy (9, 10). Alternatively, transcriptional down-regulation of the glutamate transporter (Excitatory Amino Acid Transporter2, or “EAAT2”) activity resulting in an ˜60% reduction in glutamate uptake occurs in astrocytes infected by HIV-1 or by gp120 envelope protein treatment, which might promote neuronal death by glutamate excitotoxicity (11, 12).

HIV-1 infection or gp120 treatment of human astrocytes alters gene expression patterns in vivo and in vitro (13-16). A rapid subtraction hybridization (“RaSH”) approach identified 15 astrocyte elevated genes (“AEGs”) and 10 astrocyte suppressed genes (“ASGs”) in primary human fetal astrocytes (“PHFA”) upon HIV infection (13-15). Among the AEGs, 13 were known genes and two, AEG-1 and AEG-11, were unknown at the time of cloning, while 8 of the ASGs were known and two, ASG-1 and ASG-8, were unrecognized (13-15, and International Patent Application No. PCT/US2003/011887, Publication No. WO 2004/016732, by The Trustees of Columbia University in the City of New York, Fisher et al. inventors, published Feb. 26, 2004). Analysis of the changes in gene expression pattern of known AEGs and ASGs correlated with the physiological changes observed in astrocytes following HIV infection (14-16).

### 3. SUMMARY OF THE INVENTION

The present invention is based, at least in part, on the discovery that Astrocyte Elevated Gene-1 (“AEG-1”) expression (i) suppresses the Excitatory Amino Acid Transporter-2 (“EAAT-2”) promoter, thereby inhibiting glutamate transport; (ii) supports anchorage independent colony formation of cells, in which it is synergistic with the RAS oncogene; and (iii) is increased in a number of different malignancies. It is also based, in part, on the discoveries of the AEG-1 promoter and its increased activity in the presence of activated mutant RAS.

The present invention, in a first set of embodiments, provides for inhibitors of AEG-1.

In a second set of embodiments, the present invention provides for the AEG-1 promoter, and for expression constructs comprising the AEG-1 promoter operably linked to, and controlling expression of, a gene of interest. The gene of interest may be a therapeutic gene or an antiviral gene, so that the expression construct may be used in the therapy or prevention of HAD or malignancy, or may be a reporter gene, so that the expression construct may be used in screening systems to detect agents that inhibit AEG-1 expression.

In a third set of embodiments, the present invention provides for methods of inhibiting glutamate toxicity, comprising inhibiting the expression of AEG-1.

In a fourth set of embodiments, the present invention provides for methods of preventing or inhibiting growth or survival of malignant cells, comprising inhibiting the expression of AEG-1, optionally with concurrent or sequential inhibition of RAS activity.

In a fifth set of embodiments, the present invention provides for methods of treating a neurodegenerative condition in a subject, comprising administering, to the subject, an agent that inhibits expression of AEG-1.

In a sixth set of embodiments, the present invention provides for methods of treating HAD in a subject, comprising administering, to the subject, an agent that inhibits expression of AEG-1.

In a seventh set of embodiments, the present invention provides for methods of treating a malignancy in a subject, comprising administering, to the subject, an agent that inhibits expression of AEG-1.

In an eighth set of embodiments, the present invention provides for methods of identifying therapeutic agents that may be used to treat neurodegeneration, HAD, or malignancy, comprising determining whether a test agent prevents the suppression of the EAAT2 promoter by AEG-1, wherein inhibition of suppression has a positive correlation with therapeutic benefit.

### 4. BRIEF DESCRIPTION OF THE FIGURES

FIG. 1. Complete open reading frame technique. A. Schematic diagram of C-ORF (details in text). B. Representative C-ORF applications. C-ORF products of ISG-56, mda-9/syntenin and mda-5 are resolved in 1% agarose gel (lane 2). The size of C-ORF products is compared with RT-PCR product of each gene with common 3′ nested primer and 5′ primer from reported sequence (lane 1). Nested PCR of C-ORF with anchor primer only (lane 3). C. Application of C-ORF to AEG-1 cloning. First round AEG-1 C-ORF product is resolved as above (lane 2: GSP and lane 3: anchor primer only).

FIG. 2. A baculoviral tag-free recombinant protein production system. A. Schematic diagram of AEG-1 fusion protein with chitin binding/intein domain. B. Protein electrophoretogram in 10% SDS-PAGE of input (lane 1) and elution (lanes 2 and 3) from chitin affinity column followed by DTT-induced self-cleavage. C. Immunoreactivity of αAEG-1 antibody. Protein samples (20 μg) from transiently transfected 293 cells with pcDNA3.1-AEG-1-HA were resolved in 10% SDS-PAGE and analyzed by Western blotting. Blots were probed with either αHA antibody or αAEG-1 antibody and detected by chemiluminescence.

FIG. 3. Expression pattern of AEG-1. A. AEG-1 expression in different organs. Multiple tissue Northern blots (ClonTech) were probed with ³²P-labelled 1.6 kb AEG-1 and β-actin cDNA probes. B. AEG-1 expression in various cancer cell lines. Northern blots of the indicated RNA samples were probed with AEG-1 and GAPDH probes. HMEC, human mammary epithelial cells; NC, normal cerebellum cells. C. Western blot analysis of AEG-1 expression in selected cells with αAEG-1 antibody.

FIG. 4. Immunofluorescence microscopy of AEG-1. Subcellular localization of AEG-1 was determined in IM-PHFA as described in Materials & Methods. Calreticulin and Mito-tracker were used for specific ER and mitochondria markers, respectively.

FIG. 5. Effect of AEG-1 expression on EAAT2 promoter activity. A. A luciferase reporter vector containing full EAAT2 promoter or EAAT1 promoter was co-transfected with either pcDNA3.1 or pcDNA3.1-AEG-1-HA. Two days later, cells were harvested and luciferase activity of the protein extracts was measured. Data shown is a representative of multiple experiments. B. A luciferase reporter vector containing the full EAAT2 promoter or the EAAT1 promoter was co-transfected with the indicated expression vectors. Samples were obtained and analyzed as above. Data shown is a representative of multiple experiments.

FIG. 6. Soft agar colony formation assay. A. Colony forming ability of FM516-SV cells transiently transfected with indicated expression vectors. Data shown is average±SD of three independent transfections. B. Colony forming ability of FM516-SV cells stably transfected with AEG-1 and T24 Ha-ras. Data shown is average±SD of three independent transfections. C. Expression of AEG-1 and T24 Ha-ras in the stable transfectants determined by Northern blot analysis.

FIG. 7. Expression of AEG-1 in normal brain and glioblastoma cells and tissue. A. Northern blot analysis of AEG-1 gene expression in normal or glioblastoma cell lines. The membrane containing RNA from the cells indicated was probed with an AEG-1 cDNA probe. Normalization of signals in all lanes was achieved by probing the membrane with a GAPDH probe. N.C.=normal cerebellum, cells are described in material and methods section. B. Western blot analysis of protein extracts derived from the cells or tissues indicated. The blot was probed with a specific anti-AEG-1 antibody. Equivalency of loading was determined by probing the membrane with an anti-EF1α antibody.

FIG. 8. Effect of inhibition of AEG-1 gene expression using siRNA. A. Inhibition is shown of AEG-1 gene expression by Western blot analysis of cell lines listed. A control for protein loading was performed using an anti-EF1α antibody. B. Agar cloning efficiency of U87-MG and U251-MG cells as described in material and methods is shown. C. Effect of AEG-1 inhibition on invasiveness of cell lines indicated, through a Matrigel matrix coated tissue culture insert (Transwell™ chamber) is shown.

FIG. 9. Nucleic acid sequence of the AEG-1 Promoter (SEQ ID NO:2) indicating a putative RAS-activated region at position 474 to 491. Position 1 of the sequence corresponds to the base pair immediately upstream of the translational initiation ATG codon.

FIG. 10. Schematic representation of the AEG-1 Promoter showing the location of putative transcription factor binding sites. The promoter sequence was searched against MatInspector (www.genomatix.de) and Match (www.generegulation.com) transcription factor databases. The presence and location of predicted transcription factor binding sites identified by homology searches is indicated.

FIG. 11. H-ras can activate the human AEG-1 promoter. A. Full length AEG-1 promoter activity was determined in three pairs of cell types. Each pair was comprised of a normal immortalized line THV (human astrocyte), CREF (rat embryo fibroblast) FM 516 (human melanocyte) and a RAS transformed equivalent THR, CREF-ras and FM 516-ras respectively. Comparative activity of the human full length (2759 bp) AEG-1 promoter luciferase construct (as relative luciferase activity) under identical conditions in each pair is shown in the respective panels. Data shown is an average±SD of three independent transfections. B. An Ha-RAS expression vector was co-transfected with the AEG-1 luciferase reporter at a range of 50 to 600 ng and promoter activity was determined. Data shown is an average±SD of three independent transfections.

FIG. 12. Identification of the AEG-1 promoter region responsible for Ha-ras-mediated activation of AEG-1 transcription. Luciferase reporter activity of nested deletion constructs of the AEG-1 promoter deleted at positions 1146, 787, 515 and 350, compared to full length (2759 bp) is shown. Activity of these constructs is compared between FM 516 immortalized human melanocytes and a RAS overexpressing clone of FM 516 (FM516-ras-c17). Data shown is an average±SD of three independent transfections.

FIG. 13. Effect of PI3K, MEK and p38 MAPK inhibitors on AEG-1 promoter activity. A. Immortalized (THV) and ras transformed (THR) human astrocytes were treated with indicated amount of inhibitors and luciferase activity determined. B. CREF and CREF-ras rat embryo fibroblasts were treated with indicated amount of inhibitors and luciferase activity determined. C. Immortalized (THV) and RAS transformed (THR) human astrocytes were treated with indicated amount of inhibitors and luciferase activity determined. D. CREF and CREF-ras rat embryo fibroblasts were treated with indicated amount of inhibitors and luciferase activity determined. E. The immortalized melanocyte cell line FM516 and a stable mutant RAS overexpressing clone FM516-ras7 were treated with indicated amount of inhibitors and luciferase activity determined.

FIG. 14. Activity of the AEG-1 promoter in cell lines containing mutated RAS genes. A. Human pancreatic cancer cells were transfected with the AEG-1 luciferase reporter and luciferase activity was determined. B. The human colorectal cell line HCT116 and two related progeny lines C2 and C10 were transfected with the AEG-1 luciferase reporter and luciferase activity was determined. C. The human colorectal cell line HCT116 and two related progeny lines C2 and C10 were transfected with the AEG-1 luciferase reporter and luciferase activity was determined in the presence and absence of inhibitors.

FIG. 15. The AEG-1 protein activates its own promoter. A. Stably expressing AEG-1 clones (in FM516 cells) were transfected with the AEG-1 luciferase reporter and activity was determined. B. Deletion constructs of the AEG-1 reporter were transfected into FM516 cells stably expressing AEG-1 and luciferase activity was determined.

FIG. 16. Effect of HIV gene products Tat and Nef on AEG-1 promoter activity. Expression constructs of Tat and Nef at the indicated plasmid amount was co-transfected with the AEG-1 reporter and their effect on luciferase activity was determined.

FIG. 17. Effect of cyclic AMP on AEG-1 promoter activity. Bromo-cAMP at the indicated concentration was added to cells transfected with the AEG-1 luciferase reporter and the effect of cAMP addition on promoter activity was determined.

### 5.

### DETAILED DESCRIPTION OF THE INVENTION

For clarity, and not by way of limitation, the detailed description of the invention is divided into the following subsections:

(i) inhibitors of AEG-1;

(ii) the AEG-1 promoter and expression constructs;

(iii) methods of inhibiting glutamate toxicity;

(iv) methods of preventing or inhibiting growth of malignant cells;

(v) methods of treating neurodegenerative conditions;

(vi) methods of treating HAD;

(vii) methods of treating a malignancy by inhibiting expression of AEG-1;

(viii) methods of identifying therapeutic agents using the EAAT-2 promoter;

(ix) gene therapy using the AEG-1 promoter; and

(x) drug discovery using the AEG-1 promoter.

### 5.1 Inhibitors of AEG-1

The present invention relates to inhibitors of AEG-1. Such inhibitors can inhibit AEG-1 expression (e.g., transcription, inhibition of AEG-1 promoter activity or translation) or function (e.g. inhibition of AEG-1 protein activity). Screening methods described herein, for example as set forth in sections 5.8 and 5.10, below, may be used to identify compounds that may be used as AEG-1 inhibitors according to the invention.

In certain non-limiting embodiments, inhibitors of AEG-1 are directed to inhibition of transcription of AEG-1. Such agents inhibit activity of the AEG-1 promoter, and may be identified using screening methods which employ the AEG-1 promoter linked to a reporter gene, as described in sections 5.2 and 5.10, infra. As mutant activated RAS activates the AEG-1 promoter, one non-limiting example of a class of inhibitors of AEG-1 transcription is molecules that inhibit RAS activity, such as, for example, molecules that inhibit translation of RAS, such as anti-RAS antisense RNA, interfering RNA, ribozymes or “DNAzymes.” Non-limiting examples of anti-RAS agents are set forth in International Patent Application No. PCT/JUS02/26454 by the Trustees of Columbia University in the City of New York, by Fisher, Published as W003016499 on Feb. 27, 2003.

In other non-limiting embodiments, translation of AEG-1-encoding mRNA is inhibited using a nucleic acid molecule that is, at least in part, complementary to and/or hybridizable to the AEG-1 coding sequence, such as an antisense nucleic acid, an interfering RNA (“siRNA or RNAi”), a ribozyme, or a DNAzyme. The portion complementary to AEG-1 mRNA may be between about 5-10, 5-50, 5-100, 10-50, 10-100, 20-50, 20-100, 100-500, 500-1000, 1000-2000, or 2000-3611 nucleotides in length. The nucleic acid sequence of AEG-1 is set forth in International Patent Application No. PCT/US2003/011887, Publication No. WO 2004/016732, by The Trustees of Columbia University in the City of New York, Fisher et al. inventors, published Feb. 26, 2004, in GenBank Accession No. AF411226, and is provided herein as SEQ ID NO:1. “Complementary” molecules are those which contain complementary bases to at least 90 percent, and preferably at least 95 percent, and, in non-limiting embodiments, 100 percent of the nucleotides in a subsequence of AEG-1. “Hybridizable” nucleic acid molecules hybridize to the AEG-1 gene or cDNA (sense strand) under stringent conditions. Stringent conditions, as defined herein, are hybridization to filter-bound DNA or RNA in 0.5 M NaHPO4, 7% sodium dodecyl sulfate (SDS), 1 mM EDTA at 65° C., and washing twice or more in 0.1×SSC (15-30 mM NaCl, 1.5-3 mM sodium citrate, pH 7.0)/0.1% SDS at 68° C. For DNA or RNA samples immobilized on nylon filters, a stringent hybridization washing solution may alternatively be comprised of 40 mM NaPO4, pH 7.2, 1-2% SDS and 1 mM EDTA, for which a washing temperature of at least 65-68° C. is recommended.

In one non-limiting embodiment, an siRNA comprises the sequence 5′-AGCAGCCACCAGAGATTGA-3′ (SEQ ID NO:3), or a sequence that is at least 90 or 95 percent homologous thereto, or that hybridizes under stringent conditions, and is up to 19, 25, 35, or 50 nucleotides in length.

In a non-limiting embodiment, an inhibitor of AEG-1 may comprise an antibody which specifically recognizes the AEG-1 protein and inhibits its activity. The inhibition of AEG-1 protein activity may be due to binding of the antibody to the AEG-1 protein so as to prevent access of other molecules required for or mediating AEG-1 activity. The inhibition of AEG-1 protein activity may be due to binding of the antibody to an active site in the AEG-1 protein thus inhibiting its activity, even if access to other parts of the AEG-1 protein is not blocked. In further embodiments, the antibody may be a polyclonal antibody, a monoclonal antibody, a single chain antibody, an antibody fused to another protein which possesses additional catalytic activity etc.

In another non-limiting embodiment, an AEG-1 inhibitor may comprise a single or mixture of several peptides corresponding to regions or functional subdomains of the AEG-1 protein. An AEG-1 inhibitor may also comprise an AEG-1 peptide containing one or several point mutations that impair or reverse the activity of the unmodified protein. The presence of such inhibitory peptides in proximity to the full length functional AEG-1 protein causes partial or full inhibition of the activity of the AEG-1 protein. Such partial mutant forms or an independently expressed subdomain of a protein which interferes with the biological or catalytic activity of a full length protein has usually been designated the term “dominant-negative” in the published literature. Therefore the term “dominant-negative AEG-1 protein” as used herein will encompass the properties embodied by an altered form of AEG-1 protein which interferes with the activity of the unmodified form of AEG-1 protein. In a non-limiting embodiment, the AEG-1 derived inhibitory peptides may have a range of between 10 to 900 amino acid residues.

A dominant-negative AEG-1 protein may be used in protein/peptide therapy of a subject in need of such treatment. As such, the dominant-negative AEG-1 protein of the invention may be prepared by chemical synthesis or recombinant DNA techniques, purified by methods known in the art, and then administered to a subject in need of such treatment. Dominant-negative AEG-1 protein may be comprised, for example, in solution, in suspension, and/or in a carrier particle such as microparticles, nanoparticles, liposomes, or other protein-stabilizing formulations known in the art. In a non-limiting specific example, formulations of dominant-negative AEG-1 protein may stabilized by addition of zinc and/or protamine stabilizers as in the case of certain types of insulin formulations. Alternatively, in specific non-limiting embodiments, dominant-negative AEG-1 protein may be linked covalently or non-covalently, to a carrier protein which is preferably non-immunogenic.

In a further non-limiting embodiment, the inhibitory peptide may be delivered to a cell in purified form, in a pharmaceutical formulation or by means of an expression vector. Non-limiting examples of expression vectors used to deliver inhibitory forms of AEG-1 peptides include DNA plasmid vectors, adenoviral vectors, adeno-associated virus vectors, retroviral vectors, lentiviral vectors etc.

### 5.2 The AEG-1 Promoter and Expression Constructs

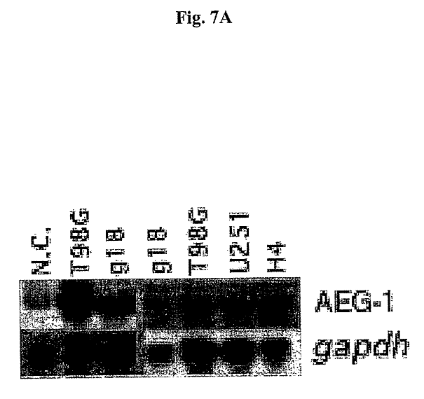

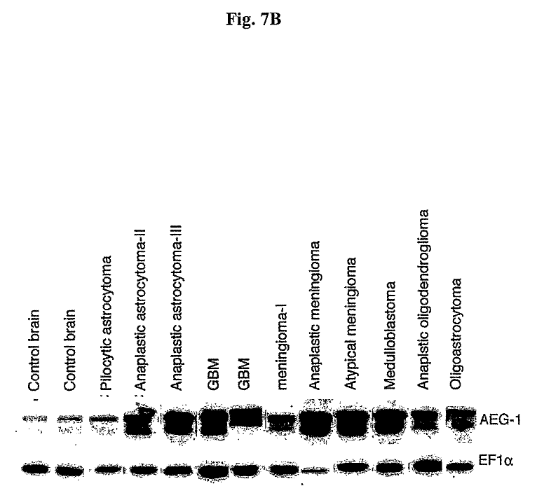

In a second set of embodiments, the present invention provides for the AEG-1 promoter. An “AEG-1 promoter,” as defined herein, includes a nucleic acid molecule having a sequence as depicted in FIG. 7 (SEQ ID NO:2), as well as nucleic acid molecules that are at least 80, 85, 90 or 95 percent homologous to SEQ ID NO: 2 (as determined using standard homology-determining software, such as BLAST or FASTA), and molecules that hybridize to a nucleic acid molecule having SEQ ID NO:2 under stringent hybridization conditions, as defined above.

The present invention further provides for a “core-AEG-1 promoter” which retains RAS sensitivity, and which comprises up to 2300, 2500 or 2600 nucleotides of SEQ ID NO:2 and at least nucleotides 1 to 515 of SEQ ID NO:2. The present invention yet further provides for the nucleic acids that are at least 80, 85, 90 or 95 percent homologous to said core AEG-1 promoter, or hybridize thereto under stringent conditions, and to vectors comprising the core AEG-1 promoter optionally linked to a gene of interest.

An AEG-1 promoter of the invention may be comprised in a vector molecule, and may be operably linked to any gene of interest. Such linkage may be used to effect selective expression of the gene of interest in astrocytes infected with HIV and in certain types of cancer cells, such as glioblastoma multiforme, astrocytoma, breast cancer, colon cancer, pancreatic cancer, and melanoma cells, and cells of cancers having an activating mutation in RAS. Vectors comprising an AEG-1 promoter operably linked to any gene of interest include but are not limited to DNA plasmid vectors, adenoviral vectors, adeno-associated virus vectors, retroviral vectors, lentiviral vectors etc.

In a specific non-limiting embodiment, an AEG-1 promoter may be operably linked to the E1A and E1B genes in a replicative adenoviral vector. Such linkage is expected to enhance the replication and cytotoxicity of the said adenovirus since AEG-1 promoter activity is increased in the presence of AEG-1 gene product. Thus enhance efficacy and specificity may be achieved in killing cancer cells expressing high amounts of AEG-1 protein. In another non-limiting embodiment the AEG-1 promoter driven replicating adenovirus may also contain an additional therapeutic gene in the E3 region.

Suitable genes of interest include therapeutic genes, antiviral genes, as well as reporter genes. A therapeutic gene is defined as a nucleic acid molecule that encodes a product, such as a protein, that confers a therapeutic benefit. A reporter gene is defined as a nucleic acid molecule that encodes a detectable product.

Suitable therapeutic genes include, but are not limited to, a gene that augments immunity, such as IFN-α, IFN-β, IFN-γ, IL-2, IL-4, IL-12 etc., a gene that has an anti-cancer effect, including genes with anti-proliferative activity, anti-glutamate activity, anti-metastatic activity, anti-angiogenic activity, or pro-apoptotic activity, such as mda-7/IL-24 (Sarkar et al. (2002) Biotechniques Suppl: 30-39; Fisher et al. (2003) Cancer Biol Ther 2:S23-37), p53 (Haupt et al., Semin Cancer Biol. 2004 14(4):244-252; Haupt et al. Cell Cycle (2004) 3(7):912-916), EAAT2 (Zagami et al., Neurotox Res. 2004;7(1-2):143-149; Huang et al., J Neurosci. 2004 24(19):4551-4559), TNF-α (Anderson et al. Curr Opin Pharmacol (2004) 4(4):314-320), IFN-β (Yoshida et al, (2004) Cancer Sci 95(11):858-865), BAX (Chan et al. Clin Exp Pharmacol Physiol (2004) 31(3):119-128), PTEN (Sansal et al. J Clin Oncol (2004) 22(14):2954-63), soluble fibroblast growth factor receptor (sFGFR) (Gowardhan et al. (2004) Prostate 61(1):50-59), RNAi or antisense-RAS (Liu et al. Cancer Gene Ther (2004) 11(11):748-756.), RNAi or antisense VEGF (Qui et al. Hepatobiliary Pancreat Dis Int (2004) 3(4):552-557), antisense or RNAi mda-9/syntenin (Sarkar et al. Pharmacol Ther (2004) 104(2): 101-115) etc.

Suitable antiviral genes include, but are not limited to, a gene that augments immunity and/or inhibits viral replication, such as IFN-α, IFN-β, IFN-γ, IL-2, IL-4, IL-12 etc.

Suitable reporter genes include, but are not limited to, luciferase, green fluorescent protein, red fluorescent protein, yellow fluorescent protein, blue fluorescent protein, beta-galactosidase, beta-glucuronidase, etc.

### 5.3 Method of Inhibiting Glutamate Toxicity

In a third set of embodiments, the present invention provides for a method of inhibiting glutamate toxicity, comprising inhibiting the expression of AEG-1. It has been observed that ectopic expression of AEG-1 specifically inhibited EAAT2 promoter activity (˜3.5-fold), where EAAT2 functions to prevent an extracellular accumulation of toxic glutamate in proximity to neurons.

Conditions in which inhibition of glutamate toxicity may be useful include neurodegenerative conditions such as but are not limited to Alzheimer's disease, Parkinson's disease, amyotrophic lateral sclerosis, and epilepsy, cerebral ischemia with or without infarction (including but not limited to cerebral infarction and transient ischemic attacks), and HAD.

Inhibitors of AEG-1 in this embodiment include those which cause inhibition of AEG-1 expression (e.g., transcription, inhibition of AEG-1 promoter activity or translation) or AEG-1 function (e.g. inhibition of protein activity) as described in section 5.1, supra. In a further embodiment, screening methods described herein, for example as set forth in sections 5.8 and 5.10, below, may be used to identify compounds that may also be used as AEG-1 inhibitors according to the invention.

### 5.4 Methods of Preventing or Inhibiting Growth of Malignant Cells

In a fourth set of embodiments, the present invention provides for a method of preventing or inhibiting growth and/or proliferation of malignant cells, comprising inhibiting the expression of AEG-1, optionally with concurrent or sequential inhibition of RAS activity. These embodiments are supported by working examples set forth in Section 6, below, as follows:

AEG-1 expression was elevated in adult astrocytes transformed by sequential expression of SV40 T/t antigen, telomerase (hTERT) and T24 Ha-ras (22) and, thereby, displaying an aggressive glioma-like phenotype;

Expression of AEG-1 promoted anchorage independent growth of FM516-SV cells that could not normally form colonies in soft agar and, although expression of the T24-Ha-ras oncogene in FM516-SV cells resulted in a higher rate in agar growth, co-expression of AEG-1 and Ha-ras in FM516-SV cells resulted in a synergistic increase in colony formation in soft agar;

AEG-1 exhibited elevated expression in many cancers, including malignant gliomas, melanomas, pilocytic astrocytoma, anaplastic astrocytoma, anaplastic meningioma, medulloblastoma, oligoastrocytoma, anaplactic oligodendroglioma and carcinomas of the breast, colon and pancreas; and

Northern blot analysis of AEG-1 expression in human cancer cells identified a different set of transcripts (9 kb, 4 kb and 1.5 kb) that might result from alternative splicing.

Accordingly, the present invention provides for a method for preventing or inhibiting the survival and or/proliferation of a malignant cell comprising administering, to the cell, an effective amount of an AEG-1 inhibitor as described above. In preferred non-limiting embodiments, the cancer cell is a glioblastoma multiforme cell, a breast cancer cell, a melanoma cell, a colon cancer cell, or a pancreatic cancer cell. In additional non-limiting embodiments, such method may be combined, either concurrently or sequentially, with administration of an anti-RAS agent (as described above).

### 5.5 Methods of Treating Neurodegenerative Conditions

In a fifth set of embodiments, the present invention provides for a method of treating a neurodegenerative condition in a subject, comprising administering, to the subject, an effective amount of an agent that inhibits expression of AEG-1. Such inhibitors can inhibit AEG-1 expression (e.g., transcription, inhibition of AEG-1 promoter activity or translation) or function (e.g. inhibition of AEG-1 protein activity) as set forth in sections 5.1. Screening methods described herein, for example as set forth in sections 5.8 and 5.10, below, may be used to identify compounds that may be used as AEG-1 inhibitors according to the invention.

Conditions which may be treated include, but are not limited to, Alzheimer's disease, Parkinson's disease, amyotrophic lateral sclerosis, and epilepsy, cerebral ischemia with or without infarction (including but not limited to cerebral infarction and transient ischemic attacks), and HAD.

### 5.6 Methods of Treating HAD

In a sixth set of embodiments, the present invention provides for a method of treating HAD in a subject, comprising administering, to the subject, an agent that inhibits expression of AEG-1. Such inhibitors can inhibit AEG-1 expression (e.g., transcription, inhibition of AEG-1 promoter activity or translation) or function (e.g. inhibition of AEG-1 protein activity) as set forth in sections 5.1. Screening methods described herein, for example as set forth in sections 5.8 and 5.10, below, may be used to identify compounds that may be used as AEG-1 inhibitors according to the invention.

### 5.7 Methods of Treating a Malignancy by Inhibiting Expression of AEG-1

In a seventh set of embodiments, the present invention provides for a method of treating a malignancy in a subject, comprising administering, to the subject, an agent that inhibits expression of AEG-1.

Such inhibitors can inhibit AEG-1 expression (e.g., transcription, inhibition of AEG-1 promoter activity or translation) or function (e.g. inhibition of AEG-1 protein activity) as set forth in sections 5.1 above. Screening methods described herein, for example as set forth in sections 5.8 and 5.10, below, may be used to identify compounds that may be used as AEG-1 inhibitors according to the invention.

Malignancies that may be treated include, but are not limited to, glioblastoma multiforme, melanoma, breast cancer, colon cancer, pancreatic cancer, and a cancer the cells of which exhibit a mutant activated form of RAS.

### 5.8 Methods of Identifying Therapeutic Agents Using the EAAT-2 Promoter

In an eighth set of embodiments, the present invention provides for a method of identifying therapeutic agents that may be used to treat neurodegeneration, HAD, or malignancy, comprising determining whether a test agent prevents the suppression of the EAAT2 promoter by AEG-1, wherein inhibition of suppression has a positive correlation with therapeutic benefit.

The invention provides for an assay for identifying an agent capable of reversing or suppressing inhibition of EAAT-2 promoter activity by AEG-1, comprising (i) preparing a nucleic acid construct having an EAAT-2 promoter operably linked to a reporter gene (defined herein as a gene having a detectable product) for example, but not limited to, a luciferase gene; (ii) introducing the construct by methods known to those skilled in the art, in a transient or stable configuration, into a cell in the assay system, such as a eukaryotic cell, for example, but not limited to, an immortalized astrocyte cell; (iii) exposing the cell to an AEG-1 gene product by methods including but not limited to exposing cells or tissues containing the EAAT-2 promoter to purified AEG-1 protein or to vectors including DNA plasmids, adenoviruses, retroviruses, adeno-associated viruses, lentiviruses etc. expressing AEG-1 gene product or providing the AEG-1 gene product by transfecting a cell within the assay system (which may or may not also contain the EAAT2 promoter/reporter construct) with an AEG-1 gene operably linked to a constitutively or inducibly active promoter; (iv) exposing the cell to a test agent; (v) measuring the amount of reporter gene product produced by a cell exposed to the test agent; and (vi) comparing the amount of reporter gene product produced by a cell containing the EAAT-2 promoter and AEG-1 gene product which has been exposed to the test agent to the amount of reporter gene product produced by a cell which has not been exposed to the test agent. To provide an adequate control, the cell (generally in a cell culture or organism) exposed to the test agent and the cell not exposed to the test agent should otherwise be maintained under the same or similar conditions. An increase in the level of reporter gene product in a cell exposed to the test agent has a positive correlation with an increase in EAAT-2 promoter activity via reduction or suppression of the inhibitory activity of the AEG-1 gene product on the EAAT-2 promoter. A test agent causing such increase in the level of reporter gene product is therefore identified as having potential therapeutic activity by inhibiting the activity of the AEG-1 gene product.

In preferred non-limiting embodiments, the type of cell utilized in an assay to identify a therapeutic agent is a cancer cell. In a further non-limiting embodiment the cancer cell is drawn from a group wherein the EAAT2 promoter may also be active, comprising of but not limited to a glioblastoma multiforme cell, a breast cancer cell, a melanoma cell, a colon cancer cell, pancreatic cancer cell or a cervical cancer cell e.g. HeLa.

In additional non-limiting embodiments, such method to identify a therapeutic agent may also be combined, either concurrently or sequentially, with administration of an anti-RAS agent (as described above).

### 5.9 Gene Therapy Using the AEG-1 Promoter

An AEG-1 promoter construct operably linked to a therapeutic gene may be used to modulate cell proliferation in a subject, wherein a nucleic acid encoding the therapeutic gene driven by the AEG-1 promoter, may be introduced into a cell of the subject.

In preferred, non-limiting embodiments, the nucleic acid encoding the AEG-1 promoter construct operably linked to a therapeutic gene may be contained in an expression vector. In preferred, non-limiting embodiments, such vectors may include DNA plasmid vectors, adenoviral vectors, adeno-associated virus vectors, retroviral vectors, lentiviral vectors etc. Examples of therapeutic genes include those set forth in 5.2.

In a specific, non-limiting embodiment of the invention, a viral vector containing a nucleic acid encoding the AEG-1 promoter construct operably linked to a therapeutic gene, may be a non-replicating or replicating adenoviral vector which may be administered to a population of target cells at a multiplicity of infection (MOI) ranging from 10-100 MOI.

In another specific, non-limiting embodiment, the amount of a non-replicating or replicating adenoviral vector viral vector administered to a subject may be 1×10⁹ pfu to 1×10¹² pfU.

In specific, non-limiting embodiments, a vector containing a nucleic acid encoding the AEG-1 promoter construct operably linked to a therapeutic gene, may be introduced into a cell ex vivo and then the cell may be introduced into a subject. For example, a vector containing a nucleic acid encoding the AEG-1 promoter construct operably linked to a therapeutic gene may be introduced into a cell of a subject (for example, an irradiated tumor cell, glial cell or fibroblast) ex vivo and then the cell containing the nucleic acid may be optionally propagated and then (with its progeny) introduced into the subject.

The present invention further encompasses the use of an AEG-1 promoter construct operably linked to a therapeutic gene in combination with other forms of therapy. For example, it encompasses the use of an AEG-1 promoter construct operably linked to a therapeutic gene in combination with other agents that have an anti-proliferative effect, including, but not limited to, anti-RAS agents, radiation therapy and chemotherapeutic agents.

As a first non-limiting example, an AEG-1 promoter construct operably linked to a therapeutic gene may be administered together with a generator of free radicals (International Patent Application No. PCT/US03/28512, by Fisher et al., published as WO 04/060269 on Jul. 22, 2004 by the Trustees of Columbia University and Virginia Commonwealth University). Examples of free radical generators include, but are not limited to arsenic trioxide, NSC656240, 4-HPR, and cisplatin. Examples of ROS include but are not limited to singlet oxygen, hydrogen peroxide, superoxide anion, hydroxyl radicals, peroxynitrite, and oxidants. In preferred embodiments, the free radical generators are arsenic trioxide, NSC656240 or 4-HPR. In other preferred embodiments, the disruptor of rnitochondrial membrane potential is PK 11195.

As a second non-limiting example, an AEG-1 promoter construct operably linked to a therapeutic gene may be administered together with a regimen of radiation therapy (International Patent Application No. PCT/US03/28512, by Fisher et al., published as WO 04/060269 on Jul. 22, 2004 by the Trustees of Columbia University). In non-limiting embodiments, an AEG-1 promoter construct operably linked to a therapeutic gene may be administered together with between 2 and 100 Gy of radiation, either as a single treatment or in multiple treatments. In one specific non-limiting embodiment of the invention, one external treatment of 2 Gy may be administered each of 5 days a week for six weeks for a total of 60 Gy. If intraoperative radiation is administered, the amount administered may be between 3 and 15 Gy total, and preferably 6 Gy.

As a third non-limiting embodiment, an AEG-1 promoter construct operably linked to a therapeutic gene may be administered together with an anti-ras agent (International Patent Application No. PCT/US02/26454, by Fisher et al., published as WO 03/016499 on Feb. 27, 2003 by the Trustees of Columbia University); particularly in the treatment of a disorder of cell proliferation associated with a mutation in a ras gene. Suitable anti-ras agents include, but are not limited to, small interfering RNAs (RNAi), antisense RNA (including but not limited to oligonucleotides having phosphorothioate residues), or farnesyl transferase inhibitors.

As a fourth non-limiting embodiment, an AEG-1 promoter construct operably linked to a therapeutic gene may be administered together with a chemotherapy agent, including, but not limited to, interferon alpha, tamoxifen, cisplatin, daunorubicin, carmustine, dacarbazine, etoposide, fluorouracil, ifosfamide, methotrexate, mitomycin, mitoxanthrone HCl, vincristine, vinblastine, and adriamycin, to name a few.

As a fifth non-limiting embodiment, an AEG-1 promoter construct operably linked to a therapeutic gene may be administered together with an anti-cancer antibody, such as, but not limited to, trastuzumab (Herceptin).

Further, an AEG-1 promoter construct operably linked to a therapeutic gene may be administered together with more than one other anti-proliferative agent (e.g., free radical generator, radiation, anti-ras agent, chemotherapeutic agent, anticancer antibody, etc.).

The amounts of anti-proliferative therapy added to the dose of an AEG-1 promoter construct operably linked to a therapeutic gene may be those doses conventionally used for such therapy. Alternatively, the combination of MDA-7 (expressed by an AEG-1 promoter construct) with another form of antiproliferative therapy may allow for the use of lower doses of said antiproliferative therapy.

Expression of the therapeutic gene of interest driven by the AEG-1 promoter in susceptible cells may achieve selective growth inhibition, selective reduction or arrest of cell proliferation or selective induction of apoptosis. Susceptible cells are those which permit overexpression of the therapeutic gene of interest due to conditions including but not limited to HIV infection or expression of mutated RAS genes so as to cause selective and relatively enhanced expression of the AEG-1 promoter compared to normal cells or cells not otherwise possessing such properties as described supra.

In preferred non-limiting embodiments, the type of cells or cancers treated by a therapeutic agent utilizing an AEG-1 promoter is drawn from a group including but not limited to a glioblastoma multiforme cell, a breast cancer cell, a melanoma cell, a colon cancer cell, and a pancreatic cancer cell. In an additional non-limiting embodiment an AEG-1 promoter based therapeutic agent may also be combined, either concurrently or sequentially, with administration of an anti-RAS agent (as described above).

### 5.10 Drug Discovery Using the AEG-1 Promoter

In a tenth set of embodiments, the present invention provides for drug discovery/screening assays utilizing expression constructs comprising a reporter gene operably linked to the AEG-1 promoter (SEQ ID NO:2), for use in screening systems to detect agents that inhibit AEG-1 expression.

The invention provides for an assay for identifying an inhibitor of AEG-1 promoter activity, comprising (i) preparing a nucleic acid construct having an AEG-1 promoter operably linked to a reporter gene (defined herein as a gene having a detectable product) for example, but not limited to, a luciferase gene; (ii) introducing the construct into a cell, such as a eukaryotic cell, for example, but not limited to, an immortalized astrocyte cell or a malignant cell; (iii) exposing the cell to a test agent; (iv) measuring the amount of reporter gene product produced by a cell exposed to the test agent; and (v) comparing the amount of reporter gene product produced by a cell exposed to the test agent to the amount of reporter gene product produced by a cell containing the AEG-1 promoter carrying construct which has not been exposed to the test agent. To provide an adequate control, the cell (generally in a cell culture or organism) exposed to the test agent and the cell not exposed to the test agent should otherwise be maintained under the same or similar conditions. A decrease in the level of reporter gene product in a cell exposed to the test agent has a positive correlation with a decrease in AEG-1 promoter activity. A test agent causing such decrease in the level of reporter gene product is therefore identified as an inhibitor of AEG-1 promoter activity. Such test agent is further identified as an inhibitor of transcription of an endogenous cellular AEG-1 gene or inhibitor of transcription of an exogenously introduced AEG-1 gene in a cell or tissue of a live organism.

In preferred non-limiting embodiments, the type of cells or cancers utilized in drug discovery using an AEG-1 promoter is drawn from a group including but not limited to a glioblastoma multiforme cell, a breast cancer cell, a melanoma cell, a colon cancer cell, or a pancreatic cancer cell. In an additional non-limiting embodiment an AEG-1 promoter based screen may also be combined, either concurrently or sequentially, with administration of an anti-RAS agent (as described above).

### 6. EXAMPLE

### AEG-1 Inhibits the EAAT-2 Promoter and is Associated with Malignancies

### 6.1 Materials and Methods

Cell Lines and Culture Conditions. PHFA, hTERT-immortalized PHFA (IM-PHFA) and primary human mammary epithelial cells (HMEC) were cultured as described (17). All brain tissue derived cell lines including Immortalized Astrocytes, T98G, G18, U251, H4 etc. were cultured as described (Yacoub et al., Cancer Biol Ther. (2003)2(4):347-53). All other immortalized or cancer cell lines were cultured as described (18)

RNA extraction and Northern blot analysis. Total RNA was extracted and Northern blotting was performed as described (18). The cDNA probes used were full-length human AEG-1, β-actin, Ha-ras and GAPDH.

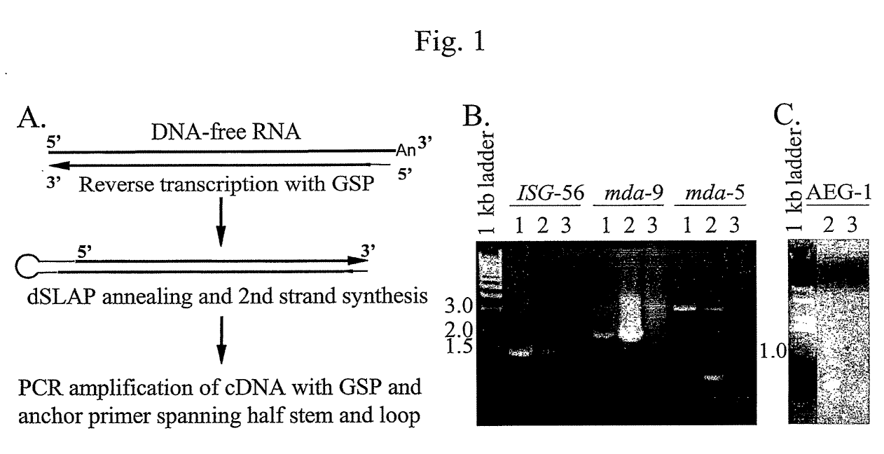

Complete Open Reading Frame (C-ORF) Technique. C-ORF consists of three steps, reverse transcription, second strand cDNA synthesis and PCR amplification of extended cDNA (FIG. 1A). RNA samples (2 μg) were reverse transcribed by SuperScript RT II (Invitrogen) with minor modifications from the manufacturer's protocol using 5 mM DTT, 2 pmole gene specific RT primer (GSP1) and 5 U RNaseOut (Invitrogen) at 45° C. First strand cDNA was purified with GlassMax (Invitrogen) after RNase H (2.2 U, Invitrogen)/RNase A digestion for 30 min at 37° C. (50 μl). A specially designed primer named dSLAP (degenerate stem-loop annealing primer, 5′ GTCTCGAGTTTAAACACTTTCTGGTCGACTAGTGTTTAAACTCGAGACN₁₂ 3′) was annealed in 20 μl using 10˜16 μl cleaned first strand cDNA, 2 pmole annealing primer and 2 μl 10×KlenTaq™ buffer [0.4 M Tricine-KOH, pH 9.2, 0.15 M KOAc, 35 mM Mg(OAc)₂ and 37.5 μg/ml BSA]. The annealing mixture was incubated at 95° C. for 1 min, gradually cooled at 5° C./min to 42° C. where it was kept for 5 min, supplemented with 5 μl polymerase mixture consisting of 0.25 μl Advantage cDNA polymerase mix, 0.5 μl 10 mM dNTPs and 0.5 μl 10×KlenTaq buffer and incubated for 30 min at 68° C. Primary PCR was performed in 25 μl reaction consisting of 5 μl of second strand synthesis reaction, 2 μl 10×KlenTaq buffer, 200 μM dNTPs, 5 pmole 3′ gene specific primer (GSP1), 10 pmole anchor primer (5′ TTCTGGTCGACTAGTGTTTAAACTCGAG 3′) and 0.25 μl Advantage cDNA polymerase mix. Basic PCR parameters that varied depending on target size, were 95° C. for 1 min., 27 cycles of 95° C. for 30 sec, 59° C. for 1 min and 68° C. for specified period (1 min for 1 kb target), and 5 or 10 min further incubation at 68° C. Nested PCR reactions (50 μl) were performed with essentially the same parameters using 0.5 μl primary PCR product, 5 μl 10×KlenTaq buffer, 0.2 mM dNTPs, 10 pmole nested GSP (GSP2), 10 pmole anchor primer and 0.5 μl Advantage cDNA polymerase mix. Single primer reaction with GSP2 only or anchor primer only was performed with primary PCR reactions to distinguish potential C-ORF artifacts. PCR reactions were resolved in 1% agarose gels and bands were purified with a gel purification kit (Qiagen). Purified bands were sequenced either with anchor primer or GSP2. Extension to the 3′ end of an expressed sequence tag (EST) was performed by RT with oligo dT adaptor primer (5′ TTCTGGTCGACTAGTGTTTAAACTCGAGACT₂₃VN 3′) (SEQ ID NO:6) followed by PCR with the anchor primer/GSP3 (5′ CTGCCTGGAGTCAAGACACTGGAGAT 3′ for AEG-1) (SEQ ID NO:7) as described above.

Construction of plasmids: Full-length AEG-1 containing a C-terminal hemagglutinin (HA)-tag was amplified by RT-PCR using primers 5′ CGGGATCCATGGCTGCACGGAGCTGGCAGGACGA 3′ (SEQ ID NO:8) and 5′ CGGGATCCCTCGAGTCACAGCGAAGCGTAGTCTGGGACGTCGTATGGGTA 3′ (SEQ ID NO:9) and an AEG-1 expression vector (pcDNA3.1-AEG-1-HA) was constructed by cloning the RT-PCR product into Bam HI site of pcDNA3.1/Hygro (+) (Invitrogen). EAAT2 and EAAT1 (GLAST) promoter-luciferase constructs were described previously (16, 17). Plasmid expressing PTEN was kindly provided by Dr. Ramon Parsons.

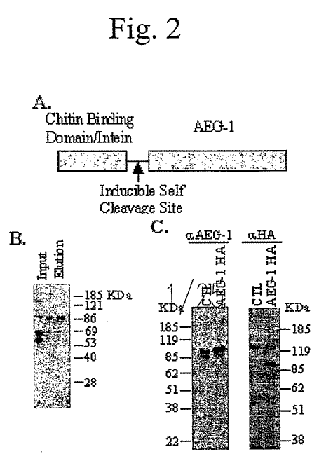

Protein synthesis and antibody production. A baculoviral transfer vector expressing intein/chitin binding domain fusion protein was constructed by substitution of GST moiety of a baculoviral vector (EcoN1/Pst1 fragment of pAcGHLT-B, Pharmingen) with intein/chitin binding domain of pTYB12 (NEB, Beverly, Mass.) obtained by PCR with 5′ TCCCCTATACTAGGTAAAATCGAAGAAGGTAAACTGGTAA 3′ and 5′ CTTCCTTTCGGGCTTTGTTAGCAGCC 3′ and digestion with EcoN1/Pst1 (pAcINT, FIG. 2A). Full ORF of AEG-1 was cloned into EcoR1/XhoI site of pAcINT (pAcINT-AEG) and transfected into Sf9 cells with BaculoGold® DNA using the manufacturer's protocol (Pharmingen). Baculovirus expressing AEG-1 was generated, and amplified in Sf9 cells. The infected insect cell extract was subjected to chitin-affinity column (NEB) and AEG-1 protein devoid of chitin binding domain was obtained by incubation of the column in 50 mM DTT that induced intein-mediated protein self-cleavage as suggested by the manufacturer (FIG. 2B, NEB). An anti-AEG-1 antibody was generated by immunization of chicken with purified AEG-1 protein (Genetel Laboratories, Madison, Wis.).

Western blotting and immunofluorescence microscopy. Whole cell lysates were prepared and Western blotting was performed as described (17). Antibodies used in specific experiments are described in the text or in corresponding figure legends. For immunofluorescence microscopy, IM-PHFA, grown on chamber slides, were probed with anti-AEG-1 and anti-calreticulin primary antibodies and FITC-conjugated anti-chicken and rhodamine-conjugated anti-rabbit secondary antibodies, respectively. Alternatively, IM-PHFA were loaded with Mito-Tracker (Molecular probes) and then immunocytochemistry was performed using anti-AEG-1 antibody and rhodamine-conjugated anti-chicken secondary antibody. Cells were visualized using a Zeiss confocal laser scanning microscope (LSM510) and a 100× objective.

Transient transfection and luciferase assay. PHFA, IM-PHFA or H4 cells were plated on 12-well plate a day prior to transfection. Cells were co-transfected using the calcium phosphate method (17). Luciferase activity was measured and normalized against protein amount as described (17).

Soft agar colony formation and Matrigel invasion assay. FM-516SV cells (2×10⁵ cells/6-cm plate) plated the previous day were transfected with either pcDNA3.1, pcDNA3.1 AEG-1, T24 Ha-ras (20 μg each), or AEG-1/T24 Ha-ras (10+10 μg). Two days later, transfected cells were harvested by trypsinization and re-plated in soft agar (0.4%) overlaying 0.8% base agar. Two weeks later, colonies over ˜50 cells in soft agar were identified microscopically and counted. Soft agar colony formation assay was also performed with FM-516SV AEG-1 stable transfectants established by transfection of pcDNA3.1 AEG-1 and T24 Ha-ras and G418 selection.

Matrigel invasion assay. Cell were cultured, transfected and harvested essentially as described above for soft-agar colony formation assays. Cells were mixed with Matrigel and plated on 0.8 μl pore-size tissue culture inserts. After 72 h or a pre-optimized time depending on the cell line, Matrigel was removed and the inserts were stained with Geimsa stain to enumerate the number of cells that had passed through the tissue culture insert membrane.

Construction of AEG-1 (AS) Adenoviral vector. The recombinant replication-defective Ad.AEG-1(AS) was created by cloning NheI-XbaI fragments from the pcDNA 3.1 vectors into pZeroTg vector in an antisense orientation. Production of infectious virus in 293 cells, analysis of recombinant virus genomes to confirm the recombinant structure, plaque purification, and titration of virus were performed by standard procedures.

Construction of AEG-1 siRNA. The sequence (5′ AGCAGCCACCAGAGATTGA 3′) (SEQ ID NO:3) used for AEG-1 silencing corresponds to the 449-467 nucleotides of the AEG-1 ORF. The siRNA was constructed with Silencer™ siRNA kit according to manufacturer's protocol. A control scrambled siRNA was used to determine specificity of the inhibition and the sequence 5′ GGGTCGTCTA TAGGGATCGAT 3′(SEQ ID NO:10)

### 6.2 Results

Complete open reading frame technology (C-ORE). C-ORF was developed to obtain full-length cDNAs from ESTs (FIG. 1A). In C-ORF, a specifically designed primer (dSLAP) anneals to the 3′ end of the reverse transcribed 1st strand cDNA and provides a primer for 2nd strand cDNA synthesis. Extended cDNA from the primer can be amplified by PCR with GSP and a universal anchor primer containing annealing primer sequence. Single round application of C-ORF resulted in full open reading frames of ISG-56 (1.5 kb) and mda-9/syntenin (2 kb) (FIG. 1B). C-ORF has been successfully applied to cloning previously unknown genes, including mda-5 (FIG. 1B), hPNPase^{old-35} and 5′ extension and identification of splice variants of PCTA-1 in a single step (19-21), which further confirms the utility of this approach. Although cDNAs cloned by single round C-ORF contained full open reading frame, they ended short by 34 nucleotides (nts) for ISG-56, 27 nts for mda-9/syntenin and 26 nts for mda-5, respectively. Premature ending of cDNA clone could be ascribed to incomplete reverse transcription reaction caused by RNA secondary structure and annealing preference of degenerate sequence in dSLAP to specific cDNA sequences such as GC rich regions. GC content of dSLAP-annealed sequences in ten C-ORF products was averaged as 70.8%, which implied certain but mild preference for GC-rich regions. However, the fact that the C-ORF products were almost full-length cDNAs suggested that second strand cDNA synthesis in the procedure preferred starting from the near-end of a cDNA rather than from the middle GC-rich sequence. The stem-loop structural motif of dSLAP that was factored in the primer design might be responsible for the preferential starting of second strand synthesis from the end of a cDNA.

Full length cloning of AEG-1. Full length AEG-1 was cloned by application of C-ORF and bioinformatics. Initially, C-ORF produced from the EST sequence corresponding to 1106-1386 nts ended up to 464 nt whose sequence matched with EST AW978779, AI816426, BF206322 and BE314632 (FIG. 1C). The 5′ end was verified by a second round of C-ORF with a different primer set and the 3′ end of AEG-1 was cloned by RT with oligo dT adaptor primer followed by PCR (data not shown). Unlike previous trials, application of C-ORF to AEG-1 ended in a shorter cDNA product. Analysis of the initial AEG-1 C-ORF product revealed that dSLAP annealed to a GC-rich sequence (GGAGGAGCCCGC). In addition, 5′ end beyond the C-ORF product had 73% GC content that suggested highly ordered secondary structure in that region (−274.02 kcal/mol for 1˜480 nts, http://ma.tbi.univie.ac.at/cgi-bin/RNAfold.cgi). Thus, both the annealing preference of dSLAP to GC-rich region and stable secondary structure in 5′ end of AEG-1 could contribute to the premature ending of AEG-1 C-ORF product. Despite success in single round cloning of full open reading frames in the case of ISG-56, mda-9/syntenin, mda-5 and other genes, repeated applications of C-ORF might be required to obtain full length cDNA depending on the specific sequence context.

Production of recombinant AEG-1 protein and Anti-AEG-1 antibody. GST-fusion protein typically used in recombinant protein production requires proteolytic digestion to remove the fused tag from the target protein. In addition, recombinant protein production in bacteria often ends in premature termination of translation and inclusion body formation. In order to minimize introduction of foreign sequences, to prevent unwanted digestion of target protein during proteolytic removal of the fusion protein and to promote consistent protein production, a hybrid baculoviral protein production system incorporating self-cleavable intein/chitin binding fusion protein system was developed and applied for tag-free AEG-1 protein production (FIG. 2A). Chitin-binding domain/intein module of the fusion protein was cleaved by treatment with 50 mM DTT after immobilization of the fusion protein on a chitin-agarose matrix (FIG. 2B). Contamination of released chitin binding domain/intein in electrophoretogram was removed by simply passing through a chitin-affinity column a second time. The electrophoretic mobility of the tag-free AEG-1 protein (Mᵣ 86) was much slower than expected from the predicted molecular mass (64 kDa), but comigrated with HA-tagged AEG-1 protein transiently expressed in HEK 293 cells. The slow mobility of AEG-1 protein is probably due to unidentified posttranslational modification or simply to the strong positive charge predicted from high pi value (9.33). Antibody against AEG-1 was raised with the purified recombinant protein in chicken and used for immunodetection (FIG. 2C). Both endogenous and transfected AEG-1-HA proteins detected by anti-HA antibody were also recognized by chicken anti-AEG-1 antibody. However, the chicken anti-AEG-1 antibody recognized an additional band of lower intensity, suggesting potential alternative splicing of the endogenous gene.

Molecular structure of AEG-1. The full-length AEG-1 cDNA consists of 3611-bp, excluding the poly A tail. The ORF from 220 to 1968 nts encodes a putative 582 amino acid protein with a calculated molecular mass of 64-KDa with a pI of 9.33. Genomic BLAST search identified AEG-1 gene consisting of 12 exons/11 introns at 8q22 where cytogenetic analysis of human gliomas indicated recurrent amplification. Protein motif analysis, such as SMART, identified a transmembrane domain, which was supported by independent transmembrane protein prediction methods (PSORT II, TMpred and HMMTOP). However, PSORT II and TMpred predicted AEG-1 as a type Ib protein (C-terminal inside), whereas TMHMM and TopPred 2 predicted type II protein (C-terminal outside). The existence of a cleavable signal peptide was not found in the motif analyses.

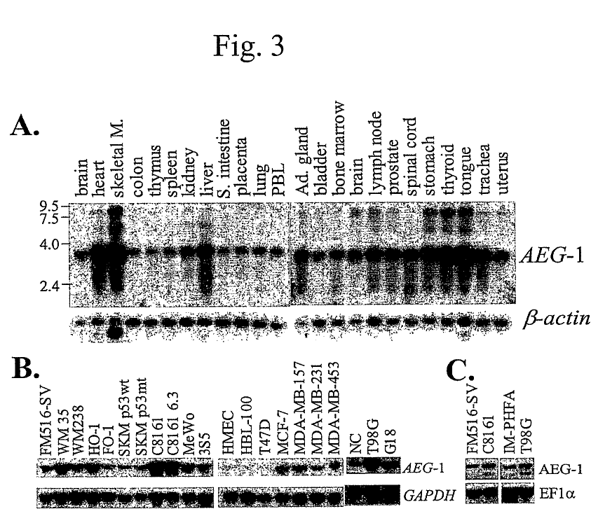

Expression pattern of AEG-1. AEG-1 expression was elevated in PHFA by 3 days post gp120 treatment and TNFα-treatment as well as HIV-1 infection and reached 3-fold over control by 7-days (13). Analysis in human Multiple Tissue Northern blots (ClonTech) demonstrated variable AEG-1 expression in all tissues (FIG. 3A). Three transcripts of 4, 6 and 9 kb that could be alternative splice variants or unprocessed/partially processed AEG-1 transcripts were detected. Interestingly, AEG-1 expression was high in two categories of organs, muscle-dominating organs such as skeletal muscle, heart, tongue and small intestine, and endocrine glands including thyroid and adrenal gland, which might suggest a role of AEG-1 in calcium-associated processes. Since chromosomal location of the AEG-1 gene (8q22) was frequently amplified in gliomas, we examined AEG-1 expression in various cancer cell lines (FIG. 3B). Northern blot analysis of AEG-1 expression in human cancer cells identified a different set of transcripts (9 kb, 4 kb and 1.5 kb) that might result from alternative splicing. FIG. 3B shows the representative band of the 4 kb transcript and it is presently not clear whether the 1.5 kb transcript, that was also reported for mouse AEG-1 (3D3/Lyric), is a specific variant in cultured cell lines (35). AEG-1 expression was up-regulated in diverse cancer cells including melanoma (HO-1, C8161 and MeWo), breast cancer (MCF-7, MDA-MB-157, -231 and -453) and glioblastoma multiforme (T98G and G18). In addition, AEG-1 expression was elevated in adult astrocytes transformed by sequential expression of SV40 T/t antigen, telomerase (hTERT) and T24 Ha-ras (22) and, thereby, displaying an aggressive glioma-like phenotype. Immunodetection of endogenous AEG-1 protein in T98G and C8161 by Western blotting (FIG. 3C) coincided with Northern blot result demonstrating the up-regulation of AEG-1 in the tumor cells over their non-transformed immortalized counterparts (IM-PHFA and FM516-SV). Although AEG-1 expression is not increased in all cancer cells, these results strongly suggest that AEG-1 may play a role in promoting cancer development or maintenance.

Expression in malignant glioma cells. AEG-1 expression is elevated in malignant glioma cell lines (such as U87 MG, U251 MG, T98G and others) as well as in human samples of brain tumors (FIGS. 7A and 7B). AEG-1 expression is also elevated in an experimental glioma model in which SV40 T/t antigen, oncogenic ras and hTERT are sequentially overexpressed.

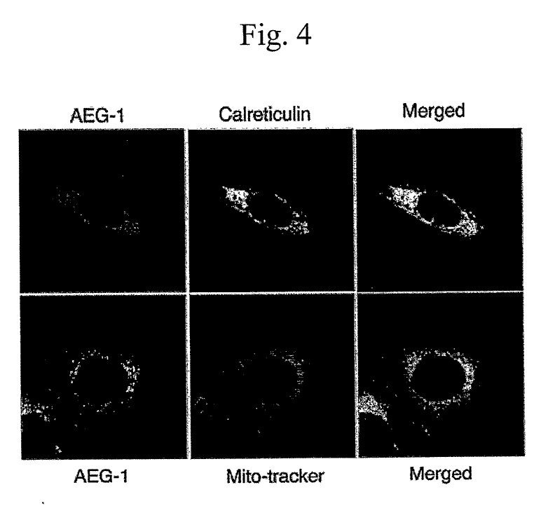

Intracellular localization of AEG-1. Intracellular localization of AEG-1 was examined in IM-PHFA by immunofluorescence microscopy with anti-AEG-1 polyclonal antibody (FIG. 4). AEG-1 was detected at the perinuclear region and in endoplasmic reticulum-like structures, but not at the plasma membrane, and co-localized with ER-specific protein calreticulin, but not with the mitochondrial marker MitoTracker. The observed ER localization of AEG-1 favors type Ib membrane topology of the AEG-1 protein. Similar localization was also observed when N-terminal or C-terminal HA-tagged AEG-1 was overexpressed and immunocytochemistry was performed with anti-HA antibody.

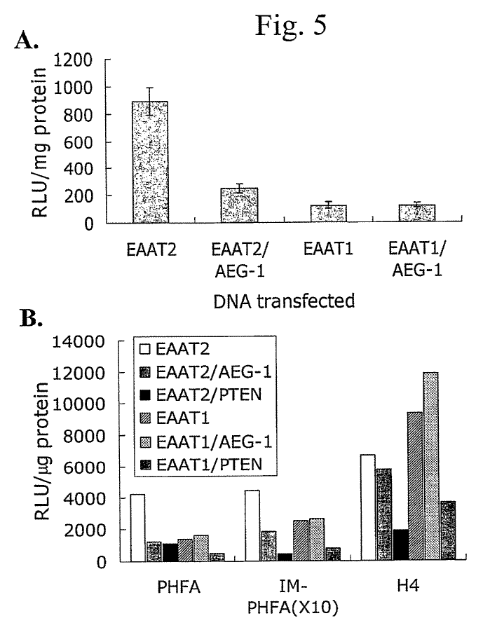

AEG-1 downregulates EAAT2 promoter activity. Infection of PHFA with HIV-1, treatment with gp120 or TNF-α resulted in elevated AEG-1 expression and decreased EAAT2 expression (11, 13, 17). The effect of AEG-1 expression on EAAT2 promoter activity was analyzed by transient co-transfection of an AEG-1 expression vector with a luciferase reporter of the EAAT2-promoter (FIG. 5A). Ectopic expression of AEG-1 specifically inhibited EAAT2 promoter activity (˜3.5-fold) in PHFA more potently than HIV-1 infection, gp120 treatment or TNFα-treatment (˜2.5-fold decrease) (17), while EAAT1 (GLAST) promoter activity was not affected by AEG-1 (FIG. 5A). AEG-1 downregulated the EAAT2-Prom activity in IM-PHFA and to a similar extent in PHFA, but not in H4 glioma cells. In contrast, expression of PTEN that suppresses PI3K signaling pathway, effectively inhibited EAAT2-Prom activity in all three-cell types (FIG. 5B). Activation of PI-3K-Akt signaling pathway was required for upregulation of EAAT2-Prom activity by EGF and cAMP (17). In this context, high EAAT2 expression in H4 cells might result from constitutive activation of the PI3K-Akt pathway (23). Taken together, AEG-1 expression induced after 3 and 7 days following HIV-1 infection, TNF-α or gp120 treatment in PHFA (13) may directly contribute to EAAT2 downregulation through a PI3K-Akt-independent pathway.

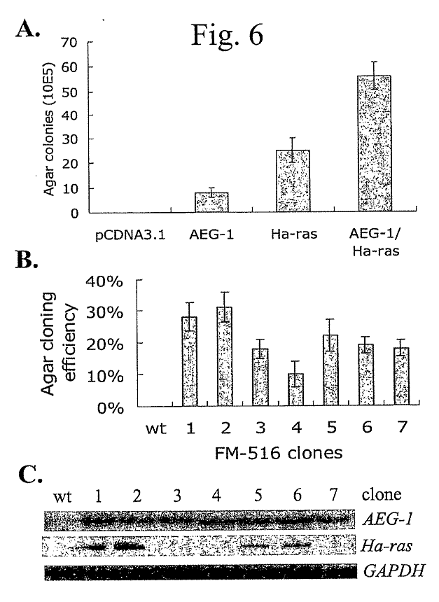

AEG-1 promotes anchorage independent growth. Steady state mRNA levels of AEG-1 are elevated in diverse cancers, including malignant gliomas, breast carcinomas and melanomas, suggesting an association between AEG-1 expression and tumorigenesis (FIG. 3C). Thus, we determined whether AEG-1 could alter tumor-associated phenotypes of nontransformed cells by soft agar colony formation assays (FIG. 6A). Expression of AEG-1 promoted anchorage independent growth of FM516-SV cells that could not normally form colonies in soft agar. Although expression of the T24-Ha-ras oncogene in FM516-SV cells resulted in a higher rate in agar growth, co-expression of AEG-1 and Ha-ras in FM516-SV cells resulted in a synergistic increase in colony formation in soft agar. FM516-SV stable transfectants co-expressing AEG-1 and Ha-ras manifested soft agar cloning ability of 10˜35% efficiency (FIG. 6B). Expression levels of Ha-ras positively correlated with the agar cloning efficiency of AEG-1/Ha-ras stable transfectants, suggesting a complementing role of AEG-1 with Ha-ras in development of the cancerous phenotype. Correlation of AEG-1 upregulation with enhanced soft agar colony forming ability was also observed in adult human astrocytes transformed by sequential expression of SV40 T antigen, hTERT and Ha-ras (THR) (22). Increased expression of AEG-1 in THR cells indirectly supports tumor-forming properties of AEG-1 in glial tumor cells. Taken together, these results strongly suggest a potential role of AEG-1 in development and progression of glioma and melanoma, since AEG-1 can synergize with Ha-ras in enhancing the transformed phenotype of specific cancer subtypes.

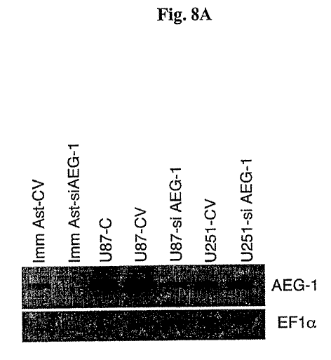

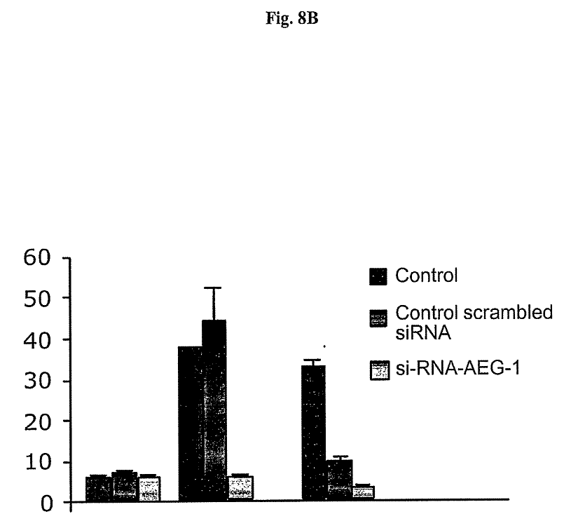

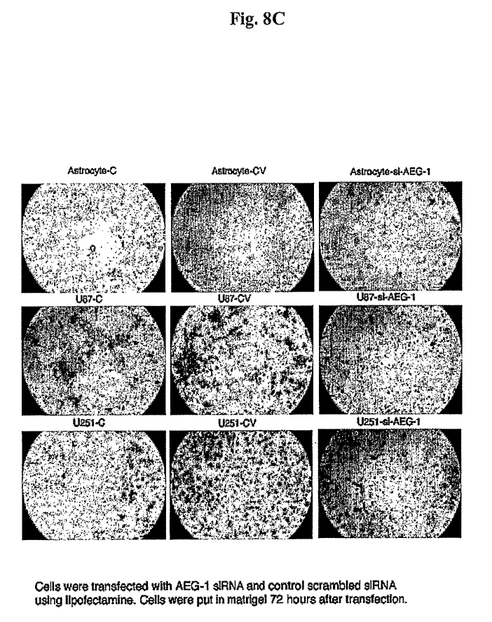

Effect of inhibition of AEG-1 on anchorage independent growth of malignant glioma cells. U87-MG and U251-MG cells were transiently transfected with siRNA for the AEG-1 gene and control scrambled siRNA using Lipofectamine. 24 hours after transfection 1×10⁵ cells from the transfection mixture were replated to determine agar cloning efficiency. In parallel, cell extracts were prepared for western blot analysis. Western blot analysis with AEG-1 antibody clearly demonstrated that efficient protein knock down was attained with the siRNA approach (FIG. 8A). siRNA for AEG-1 significantly inhibited the agar cloning efficiency in U87-MG and U251-MG cells further supporting the view that AEG-1 displays transformation-promoting activity (FIG. 8B).

Effect of inhibition of AEG-1 on invasiveness of malignant glioma cells. U87-MG and U251-MG cells were transiently transfected with siRNA for AEG-1 and with control scrambled siRNA using Lipofectamine. 24 hours after transfection cells were seeded in the upper compartment of a Transwell™ unit (Becton Dickinson) and then incubated for 72 h at 37° C. The lower compartment contained 0.5 ml of DMEM and 5% FBS. At the end of the invasion assay, the filters were removed, fixed, and stained. Invasion was determined by counting cells that had migrated to the lower side of the filter with a microscope at 100× magnification (ten fields/cell-line were counted). siRNA for the AEG-1 gene significantly inhibited Matrigel invasion in U87-MG and U251-MG (FIG. 8C) suggesting a potential role for AEG-1 in glioma invasiveness.

### 6.3 Discussion

Proof-of-principle is now provided for two technological advancements for full length cDNA cloning and tag-free recombinant protein production with documented application for the molecular and functional characterization of an HIV-induced gene, AEG-1. Significant numbers of full-length cDNAs continue to be described, the majority of which being identified using 5′ and 3′ RACE approaches. RACE methods are based on tagging the 3′ end of cDNA by terminal deoxynucleotide transferase (TdT) reaction, template-independent DNA polymerase activity of reverse transcriptase and ligation of RNA primer to 5′ end of cap-removed mRNA (24-26). However, TdT-mediated addition in original RACE often ends short and requires repeated applications to obtain a full-length cDNA. Success in gene identification is dependent on sequence context and in the RNA ligation-mediated protocol on the efficiency of cap-removal and RNA ligation reactions. In contrast, C-ORF uses a structured annealing primer in second strand cDNA synthesis that apparently promotes the reaction to start near the 3′ end of the first strand cDNA. C-ORF, although not completely free from sequence context of target genes, demonstrates successful applications in most applications using a single reaction thus making it a useful and attractive alternative in full-length cDNA cloning.

A tag-free recombinant protein production system utilizing self-cleavable intein was originally developed for use in bacterial systems (27). Application of the protocol in eukaryotic systems dispensed with modification of tRNA pools in bacteria and supports production of active soluble proteins that is often problematic in bacterial recombinant protein production. Substitution of glutathione S-transferase moiety with chitin binding domain/intein resulted in tag-free AEG-1 production without protease treatment in a baculoviral system as efficiently as in a bacterial system. Proteins produced in this way were directly used for immunization for antibody production and activity assays supporting the usefulness of this method for functional protein production.

HIV infection or gp 120 treatment of astrocytes induces substantial alterations of gene expression (14-16, 28). However, the role of these alterations in mediating physiological changes of astrocytes and consequently promoting HAD has been studied for only a limited number of genes. Elevated expression of AEG-1 in HIV-1-infected or gp120-treated astrocytes was previously demonstrated (13). Expression of AEG-1 by transient transfection inhibited EAAT2 promoter activity. The amino acid glutamate is the major mediator of excitatory signals in the mammalian CNS (29). The glutamate concentration in the extracellular fluid (ECF) determines the extent of glutamate receptor stimulation and it is of clinical importance that the extracellular glutamate concentration be kept low because glutamate is a potent neurotoxin at high concentration (30). Excitatory amino acid transporters regulate the removal of glutamate from ECF and thus protect neurons from glutamate-induced excitotoxicity (30). EAAT2, which is primarily expressed in astrocytes, is a major HIV-modulated glutamate transporter in brain and its transcription is downregulated upon HIV infection and gp120 treatment in astrocytes (16, 17). Downregulation of EAAT2 leading to glutamate excitotoxicity has been implicated as a major determinant of the pathogenesis of HAD and also of various neurodegenerative diseases including Alzheimer's disease, amyotrophic lateral sclerosis and ischemic injury (31-33). The inverse correlation of AEG-1 and EAAT2 expression by HIV infection and AEG-1 downregulation of EAAT2 promoter indicate an essential contribution of AEG-1 to the generation of HAD. Analysis of AEG-1 expression in other neurodegenerative diseases will provide a global perspective of the role of AEG-1 in the molecular pathogenesis of these disorders.

Two independent research groups recently reported human (metadherin) and mouse AEG-1 (3D3), and a rat homologue of AEG-1 (lyric) has been deposited in GenBank (not published, AF100421) (34, 35). However, not only is the context of gene identification distinct, but also the postulated functions for these homologous genes are dissimilar. In the case of the mouse orthologue of AEG-1 (3D3/lyric), cloned by a nuclear localized gene-trap screen, no association with cancer is evident, whereas rat and human AEG-1 (lyric/metadherin/AEG-1) are overexpressed in cancer cells, including hepatoma, breast cancer, glioma and melanoma, suggesting a tumorigenic and/or tumor promoting function of AEG-1. Major discordance among the AEG-1/metadherin/3D3 reports involves intracellular localization and functionality with respect to its localization. Antibody-assisted FACS analysis suggests that Metadherin has type UI membrane topology (34). However, 3D3/lyric and AEG-1 suggest type lb topology based on ER location, detected by immunofluorescence microscopy, and clusters of basic amino acids at the C-terminal juxtaposition of its transmembrane domain (35). Sequence motif analysis equivocally supports both topologies depending on the algorithm employed. However, ER, perinuclear or nuclear localization of metadherin is difficult to reconcile with its postulated role in metastasis (35) unless the localization might be cell type dependent. Therefore, the exact localization and membrane topology needs to be clarified by further analysis.

Tumorigenic potential of AEG-1 was supported by two observations, elevated expression in subsets of cancer cell lines and promotion of anchorage independent growth of immortalized melanocytes and astrocytes. Substantiation of tumorigenic potential also comes from enhanced metastasis of metadherin/AEG-1 expressing cells (34). AEG-1 may promote tumor progression indirectly, since overexpression of AEG-1 did not promote cell proliferation. Synergy with oncogenic Ha-ras that could stimulate cell proliferation further supports a more limited (potentially cooperative) role of AEG-1 in tumor formation. Anchorage-independent growth is a key component in tumor cell expansion, which might be stimulated by AEG-1 upregulation by an unidentified mechanism. In the glioma model, promotion of tumor formation might be ascribed to AEG-1-mediated EAAT2 downmodulation. Growth of glioma mass are restricted by limited intraskull space and neighboring tissue. Glutamate excitotoxicity to neighboring neurons resulting from active release of glutamate or by reduction of glutamate transporter activity and subsequent glutamate uptake is known to promote malignant glioma pathogenesis (36, 37). In fact, EAAT2 promoter activity in glioma cells, except H4, is lower by an order of magnitude than in PHFA and several fold lower than in IM-PHFA (17). It is possible that elevated levels of AEG-1 in these cells could contribute to tumorigenesis by inhibiting EAAT2 transcription and glutamate uptake.

In addition, Western blot analysis with AEG-1 antibody clearly demonstrated that efficient protein knock down could be achieved with the siRNA approach (FIG. 8A). siRNA to the AEG-1 gene significantly inhibited the agar cloning efficiency in U87-MG and U251-MG, further supporting the view that AEG-1 displays transformation-promoting activity (FIG. 8B). The above observations further demonstrate a connection between AEG-1 expression and malignancy. Confirmation of this property of AEG-1 was independently obtained by demonstrating that invasion through Matrigel is also impaired when cells are treated with an siRNA molecule which knocks down the expression level of AEG-1 (FIG. 8C)

### 7. EXAMPLE

### An Active AEG-1 Promoter Region Responsive to Oncogenic Ras-Signaling

### 7.1 Materials and Methods

Cell Lines and Culture Conditions. THV and THR immortalized human astrocytes were cultured and are described in Rich et al. (22). CREF and CREF-ras rat embryo fibroblasts and FM516 and FM516-ras human immortalized melanocytes are described in (17). All immortalized or cancer cell lines other than THV and THR were cultured as described (18).

Construction of plasmids: The full-length AEG-1 promoter DNA used in this analysis has a 3′ boundary 1 base-pair 5′ to the translation initiation site (ATG codon) of the AEG-1 gene (designated as nucleotide 1 in FIG. 7; SEQ ID NO:2) and extends 2759 bp upstream. The genomic DNA region corresponding to the AEG-1 promoter was isolated by PCR using the PCRx Enhancer System (Invitrogen, Carlsbad, Calif.) with forward primer: 5′-GGTACCCTTTAGTAATCCCTCCCTCTCT -3′ (KpnI site underlined) (SEQ ID NO:4) and reverse primer: 5′-CTCGAGATCTTCCCTCCCGTCAGAGGGACT-3′ (XhoI site underlined) 9SEQ ID NO:5). PCR conditions were as follows: Denaturation 95° C., 30 sec; annealing 55° C., 30 sec and extension 68° C., 3 min, repeated for 30 cycles. This region of genomic DNA was cloned into the luciferase reporter pGL-3 basic (Promega, Madison, Wis.) using restriction sites in KpnI and XhoI in the plasmid polylinker.

The nested 5′ deletion constructs of the AEG-1 full length promoter were constructed as follows:

The clone with a 5′ boundary at position 1146 was constructed by PCR using the forward primer 5′-GGTACCGAATTTTTGCAAACCCCTTT-3′ (KpnI site underlined) (SEQ ID NO: 13) and reverse primer 5′-CTCGAGATCTTCCCTCCCGTCAGAGGGACT-3′ (XhoI site underlined) (SEQ ID NO:14). The PCR derived fragment was ligated into the KpnI/XhoI sites of pGL3-basic.

The clone with a 5′ boundary at position 787 was constructed by isolating a SacI-XhoI fragment from pGL3-AEG1 full promoter and cloned into the SacI/XhoI site of pGL3-basic polylinker.

The clone with a 5′ boundary at position 515 was constructed by isolating a KpnI-PflMI fragment from pGL3-AEG1-787 clone. The cohesive ends made by the restriction enzyme reaction were blunt ended with DNA polymerase 1, Klenow fragment (NEB) and then ligated into pGL3-basic.

The clone with a 5′ boundary at position 350 was constructed by isolating a KpnI-BssHII fragment from the pGL3-AEGI-787 clone. The cohesive ends made by the restriction enzyme reaction were blunt ended with DNA polymerase I, Klenow fragment (NEB) and then ligated into pGL3-basic.

All deletion constructs initiated at the same 3′ end (position 1 of SEQ ID NO:2) and terminated at 350, 515, 787 and 1146 bp upstream of position 1 (FIG. 8).

Putative binding sites for transcription factors shown in FIG. 8 were determined by doing a binding-site consensus search against two transcription factor databases i.e. MatInspector (www.genomatix.de) and Match (www.generegulation.com).

Transient transfection and luciferase assay. CREF, CREF-ras, FM516, FM516-ras, THV and THR cells were plated on 24-well plates one day prior to transfection. Cells were transfected using Lipofectamine 2000 reagent under conditions recommended by the manufacturer (InVitrogen, Carlsbad, Calif.). Luciferase activity was measured and normalized against β-galactosidase activity produced by a co-transfected vector as described (17).

Cell treatment with inhibitors. The PI3 kinase inhibitor LY294002, MEK inhibitor PD 98059 and p38 MAP Kinase inhibitor SB 203580 were used to treat cells at the indicated concentration and to determine the effect of inhibition of the respective pathway on AEG-1 promoter activity using the AEG-1 luciferase reporter.

### 7.2 Results and Discussion

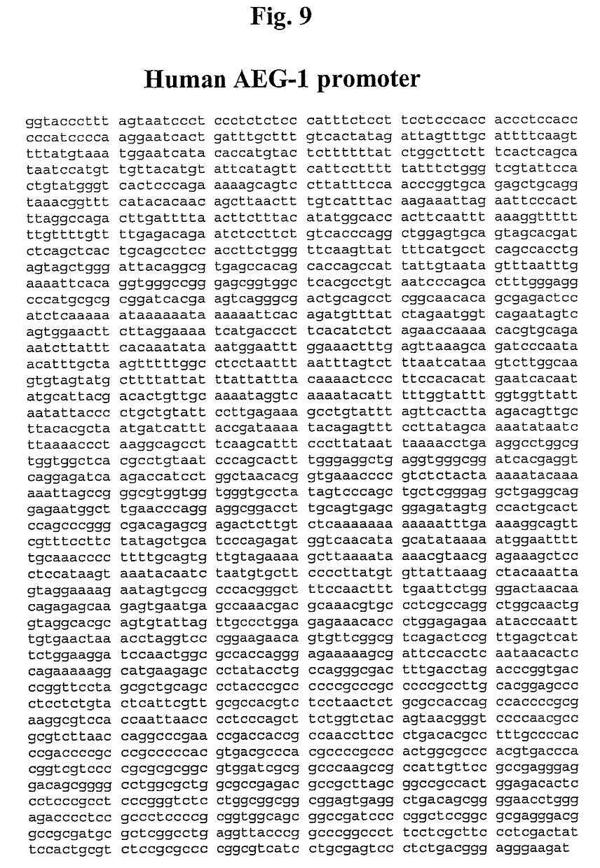

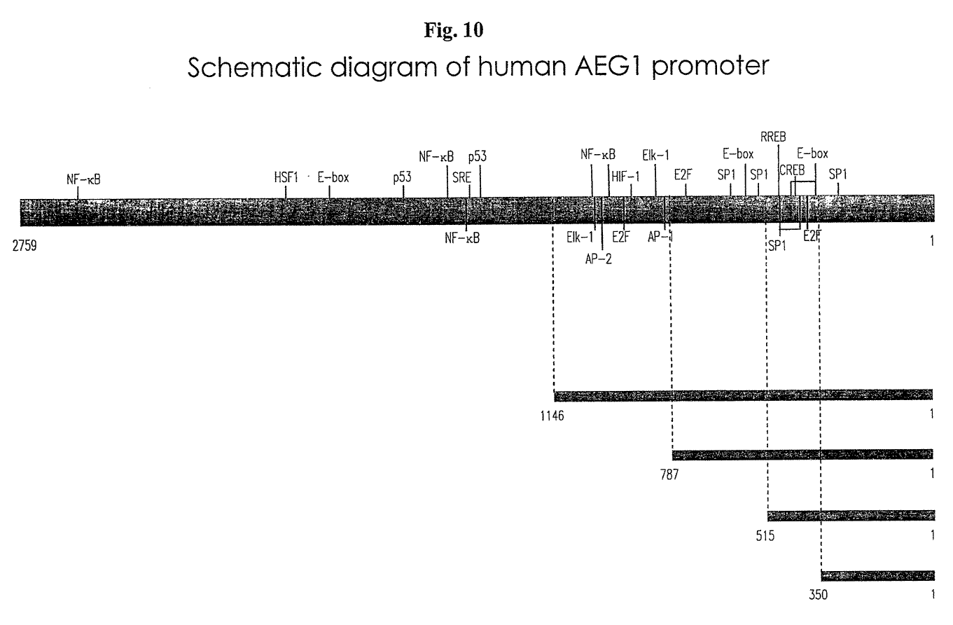

Sequence of the AEG-1 promoter and transcription factor binding sites. Human genomic DNA was PCR amplified and cloned into the luciferase reporter plasmid pGL3 as described in 7.1. The cloned DNA was sequences and analyzed for putative transcription factor binding sites. Several potential binding sites were identified and are summarized in FIG. 10. These include NF-kB, HSF1, E-box, p53, SRE, Elk-1, AP-2, HIF-1, E2F, SP1 and CREB. It appears that the promoter lacks a consensus TATA-element and therefore the AEG-1 promoter may be classified as a TATA-less promoter. In particular, a binding site for a RAS-responsive transcriptional element (RREB) (Ray et al., (2003) Mol Cell Biol 23(1): 259-271) was detected between positions 491 and 474 of the full AEG-1 promoter, FIG. 9 (SEQ ID NO:2).

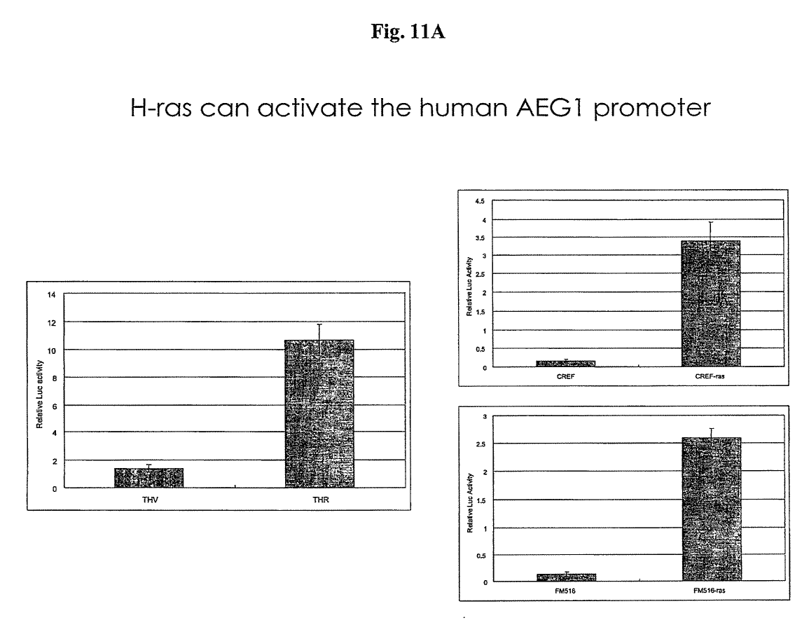

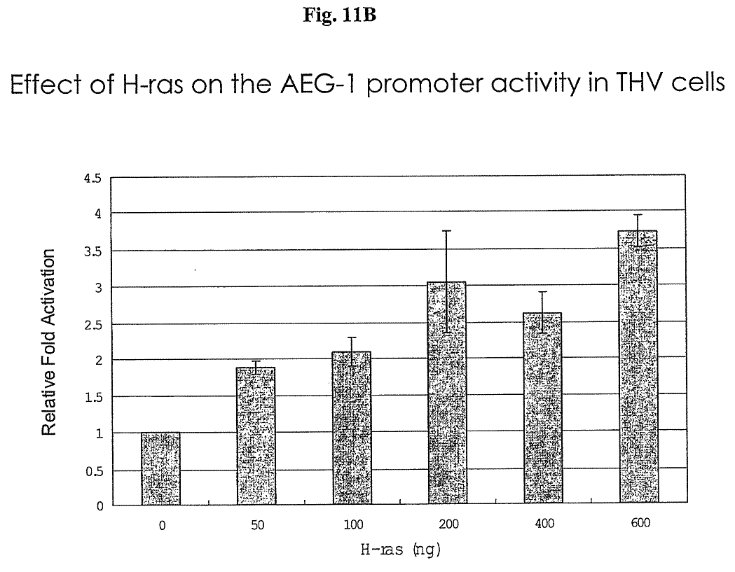

The 2759 bp genomic DNA fragment was tested for promoter activity by transient transfection into human immortalized astrocytes (THV), immortalized human melanocytes (FM516) and immortalized rat embryonal fibroblasts (CREF) and in corresponding mutated RAS-gene overexpressing clones of these cell lines i.e. THR, FM516-ras and CREF-ras (FIG. 11). Comparison of relative luciferase activity between RAS-overexpressing versus normal immortalized cells clearly indicate that the full length AEG-1 promoter shows a statistically significant transcriptional response to the activated RAS pathway. Thus greater than 5-fold increase in promoter activity is seen in THR versus THV cells; while around 12-fold increase in activity is seen in CREF-ras versus CREF cells and in FM516-ras versus FM516 cells (FIG. 11.). Similarly, introduction of a exogenous H-RAS gene into immortalized human astrocytes (THV) resulted in a dose dependent activation of the AEG-1 luciferase reporter (FIG. 11B)

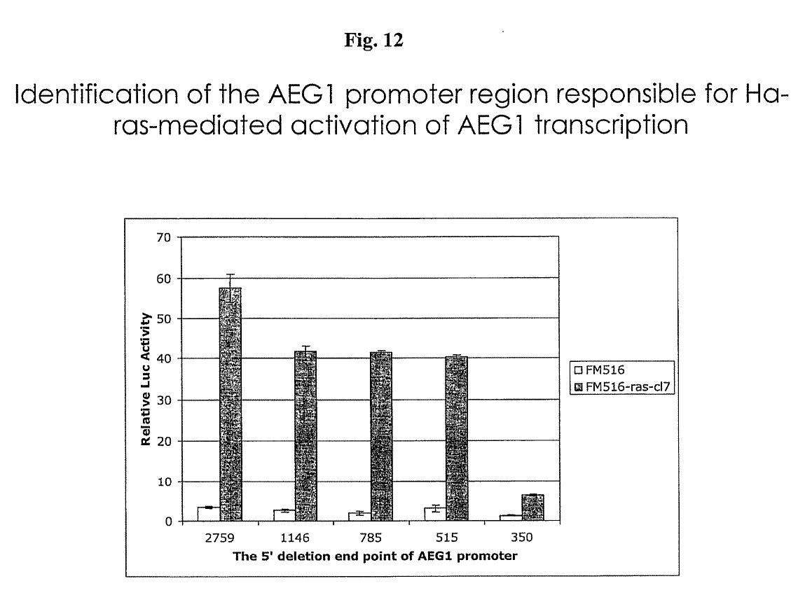

Deletion analysis of the AEG-1 promoter. Deletion analyses of the AEG-1 promoter was also performed to determine the location of potentially important regulatory regions. Nested 5′ deletions constructs of the full length 2759 bp promoter were made with cut-off points at nucleotide positions 1146, 787, 515 and 350 respectively (FIG. 10). These deletion constructs were analyzed for reporter activity compared to the full length promoter. Deletion of approximately 2400 of the 2759 bp contained in the full length promoter reduced the basal activity of the deleted promoter by approximately 2-fold. Deletions up to 515 bp from the 3′ end did not significantly affect basal activity of the promoter (FIG. 12, compare activity of the 2759 versus 515 bp fragment). However, when the deletion mutants were analyzed for RAS responsiveness in a FM-516-ras-c17 background, deletion of approximately 1600 bp (FIG. 12, compare the 2759 bp construct to the 1146, 787 and 515 constructs) resulted in about 25 percent loss of activity. Around 90% loss of RAS responsiveness was observed when the putative RREB element was deleted (FIG. 12, compare the activity of the 2759 versus the 350 bp reporter constructs). The putative RAS responsive element is located between positions 491-474. Thus the AEG-1 promoter shows strong RAS responsiveness. Based on this deletion analysis it appears that the AEG-1 promoter may comprise of a “core-promoter” region as defined by the construct with 5′ boundary at position 515 and which retains responsiveness to RAS activation. Additional transcriptional enhancer elements may reside in the region between nucleotide positions 2759 and 1146. Other transcription factors with putative binding sites in the AEG-1 promoter as shown in FIG. 10, or additional unknown factors could also play a role in the activity and responsiveness of the promoter.

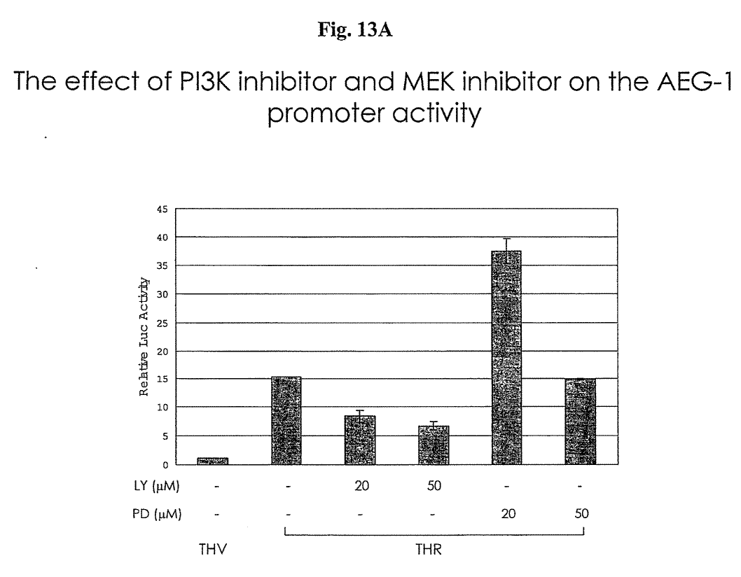

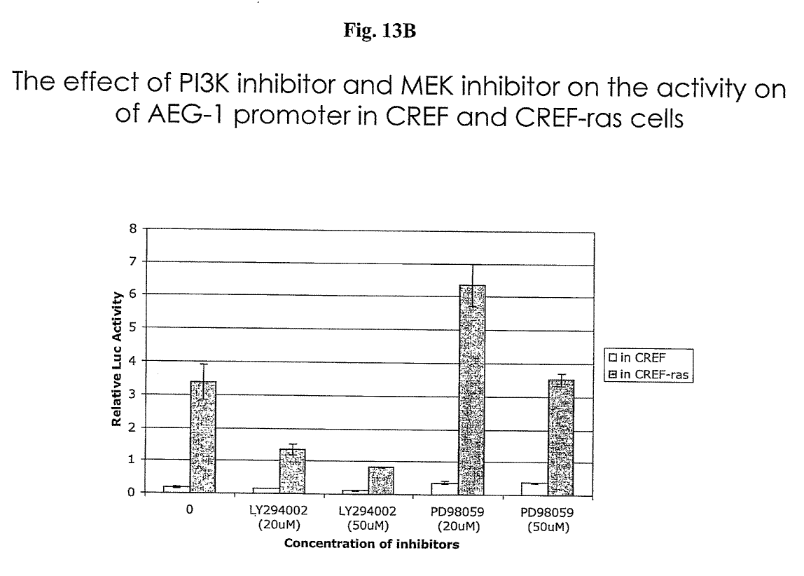

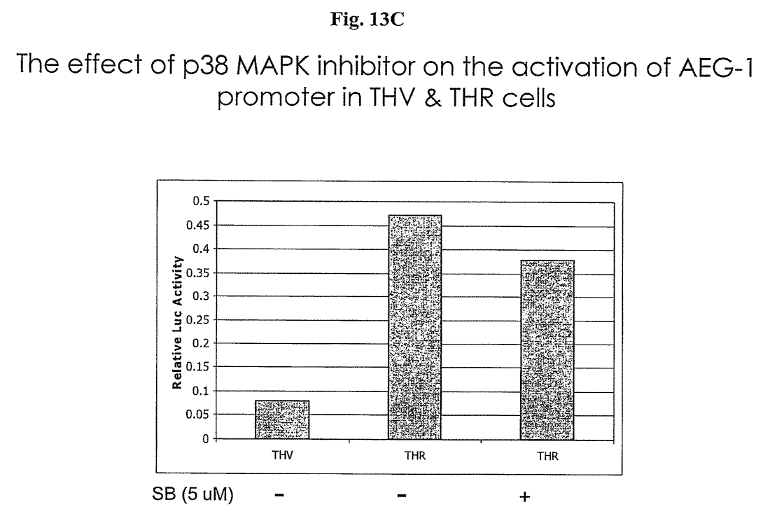

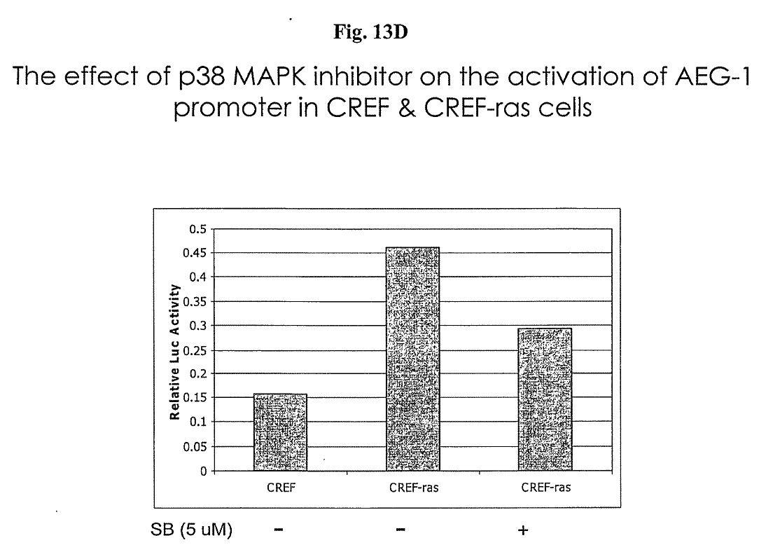

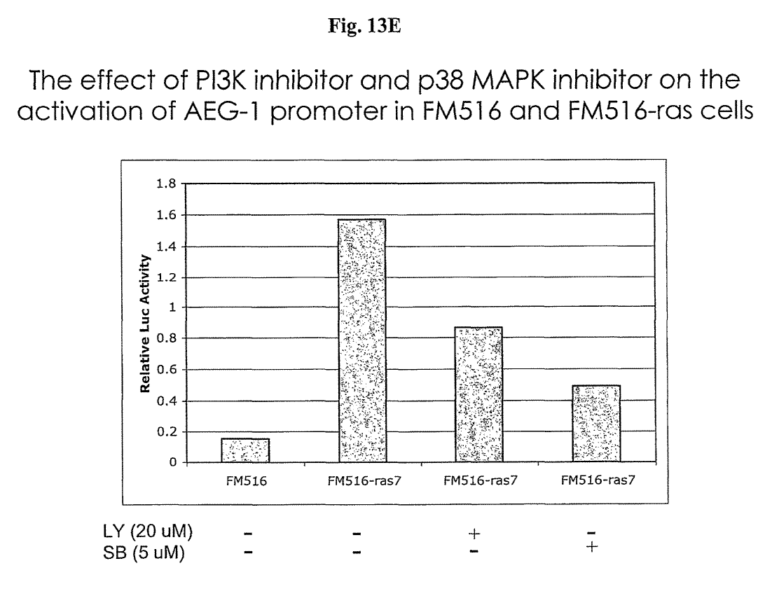

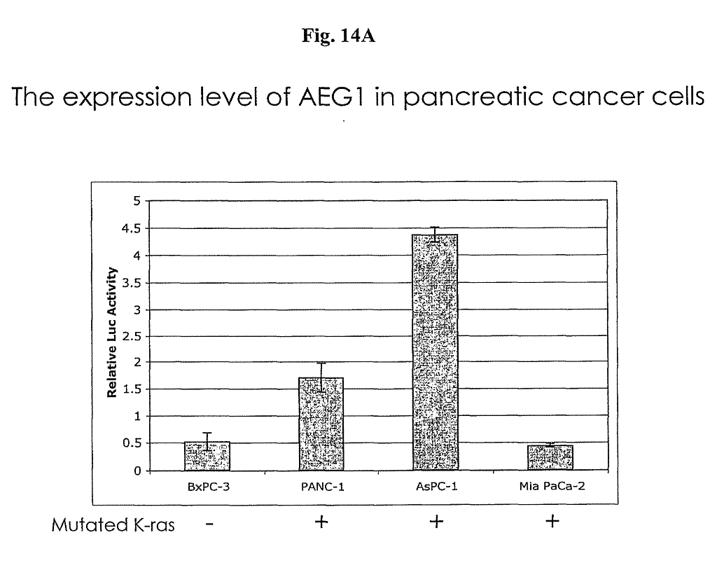

Effect of inhibition of PI3 Kinase, MEK kinase and p38 MAP kinase on AEG-1 promoter activity. Signal transduction pathways impinging on or directly downstream of the RAS pathway were analyzed to determine their effect on AEG-1 promoter activity (FIGS. 13 A-E). These analyses were performed utilizing a concentration of 20 and 50 μM of LY294002 and PD 98059 inhibitors in THV/THR astrocytes (FIG. 13. A), and CREF and CREF-ras fibroblasts (FIG. 13 B). A dose dependent inhibition of promoter activity was observed in the cell lines tested. The p38 MAPK pathway was inhibited utilizing 5 μM of SB 203580 in THV/THR astrocytes (FIG. 13C) and CREF and CREF-ras fibroblasts (FIG. 13C). Again, inhibition of promoter activity was observed in the presence of the respective inhibitor. A RAS overexpressing immortalized melanocyte line (FM516-ras7, FIG. 13E) was tested for the effect of inhibiting PI3K and p38 MAPK activity on AEG-1 promoter activity. Again, inhibition of the AEG-1 promoter was observed following treatment with inhibitor.

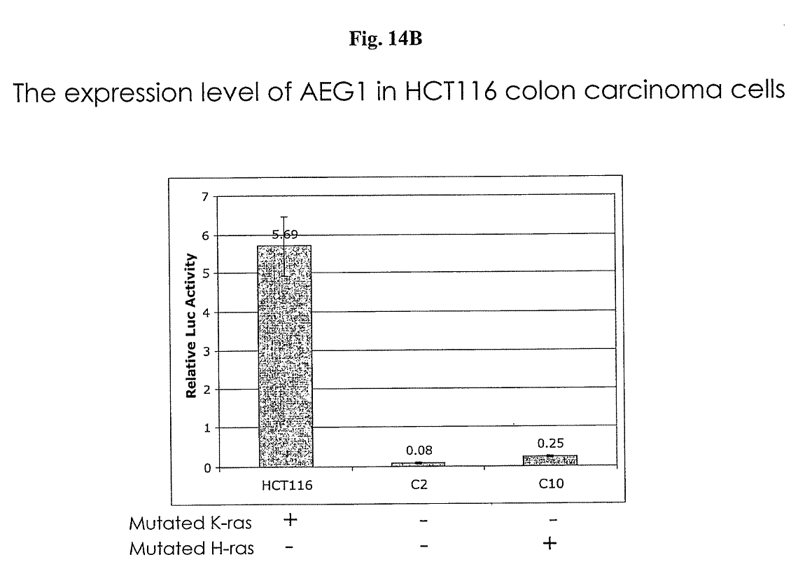

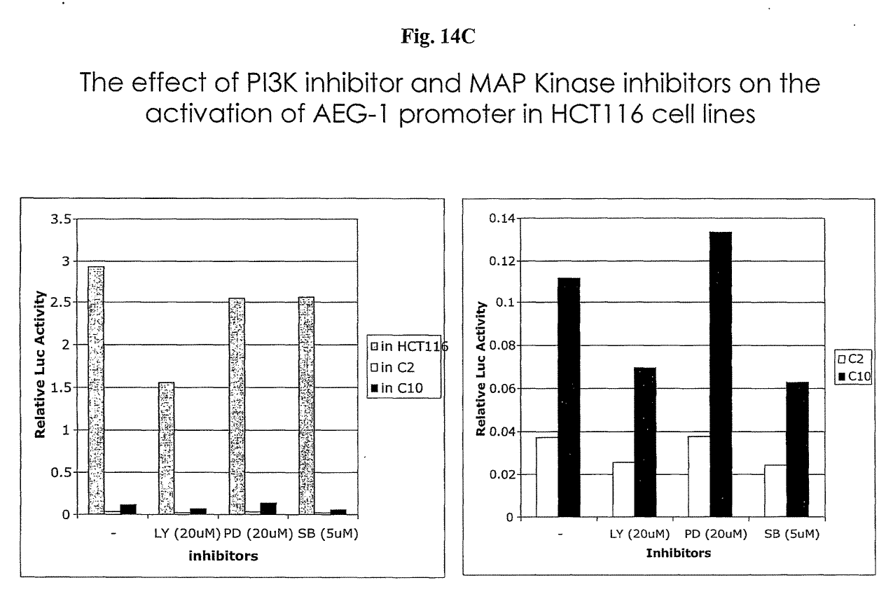

AEG-1 promoter activity in human cancer cell lines. Human cancer cell lines endogenously expressing mutated RAS genes were tested for the ability to support AEG-1 promoter activity. This contrasts with the earlier series of cells where a mutated RAS gene was exogenously introduced into the cell. The cell lines tested included the human pancreatic cancer derived cells PANC-1, AsPC-1, MiaPaCa-2 (all containing mutated k-RAS) and BxPC-3 (wild type k-RAS). PANC-1 and AsPC-1 cells supported relatively high levels of AEG-1 promoter activity while BxPC-3 and Mia PaCa-2 cells showed relatively lower transfected AEG-1 luciferase reporter activity (FIG. 14A).

The colorectal cancer cell line HCT116 which is mutated in the k-RAS gene was also tested for AEG-1 promoter activity in addition to two related cell lines. One of these lines, C2 is a clone of HCT116 somatically knocked out for the k-RAS gene and the other, C10 is a clone derived from C2 wherein a mutated Ha-RAS gene has been reintroduced into C2 so that it now expresses a mutated Ha-RAS gene instead of a mutant k-RAS gene. HCT116 cells support approximately 70-fold and 22-fold higher activity of the AEG-1 promoter compared to C2 and C10 lines respectively (FIG. 14B). These colorectal cancer derived cell lines were also tested for AEG-1 promoter activity after treatment with PI3K, MEK and p38 MAPK inhibitors as described previously. AEG-1 promoter activity in the parental HCT116 line was inhibited to a lesser extent than astrocytes and fibroblasts described in the earlier section. However some inhibition of promoter activity was observed with all inhibitors in this cell line (FIG. 14C). The C2 and C10 cell lines were however not responsive to PD 98059 and showed some responsiveness with the other two inhibitors.

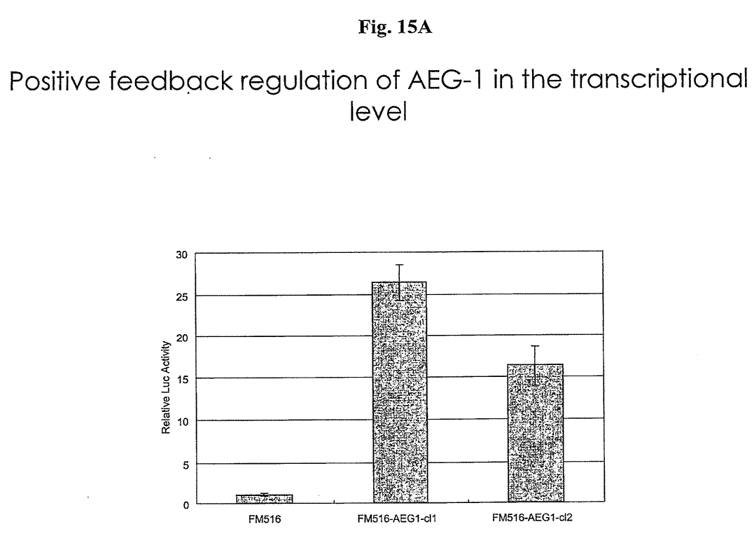

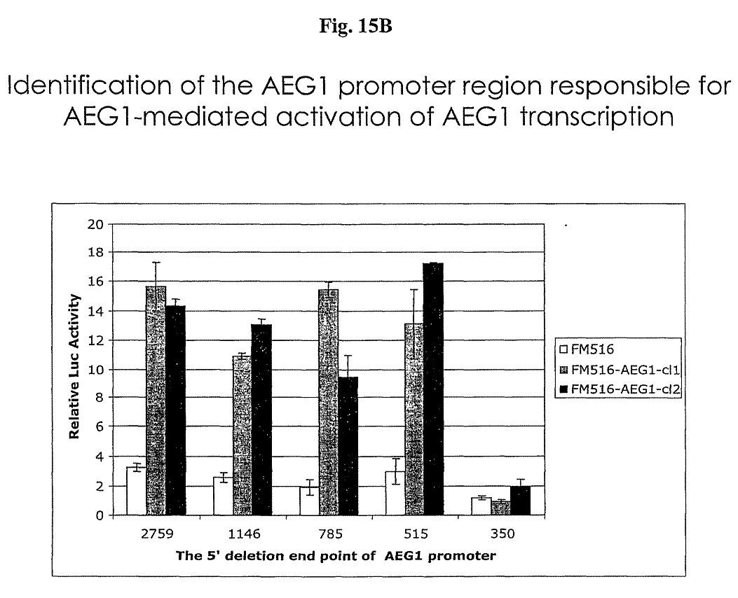

The AEG-1 gene product can activate its own promoter. Transfection of the full length AEG-1 luciferase reporter construct into a FM516 melanocyte stably expressing AEG-1 protein showed that a positive feedback loop may cause AEG-1 gene product to enhance its own expression level (FIG. 15A). This responsiveness to AEG-1 was shown by all deletion constructs described previously except the reporter deletion containing only nucleotides 1 to 350 of the AEG-1 promoter (FIG. 15B).

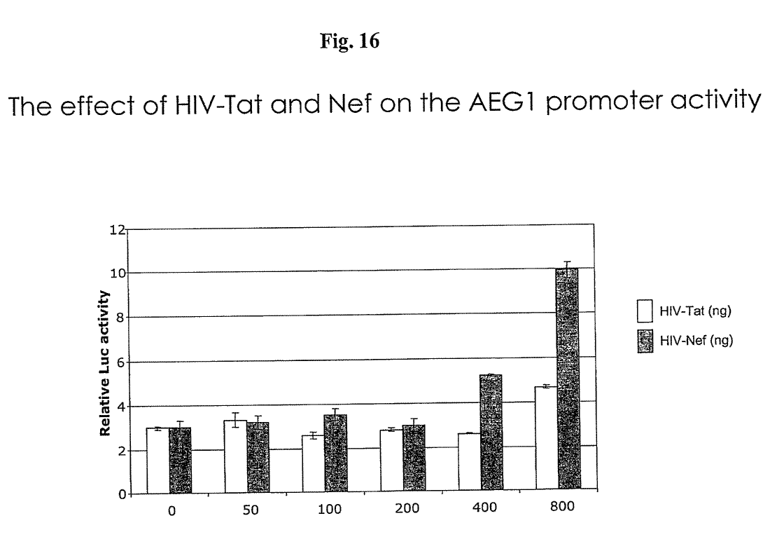

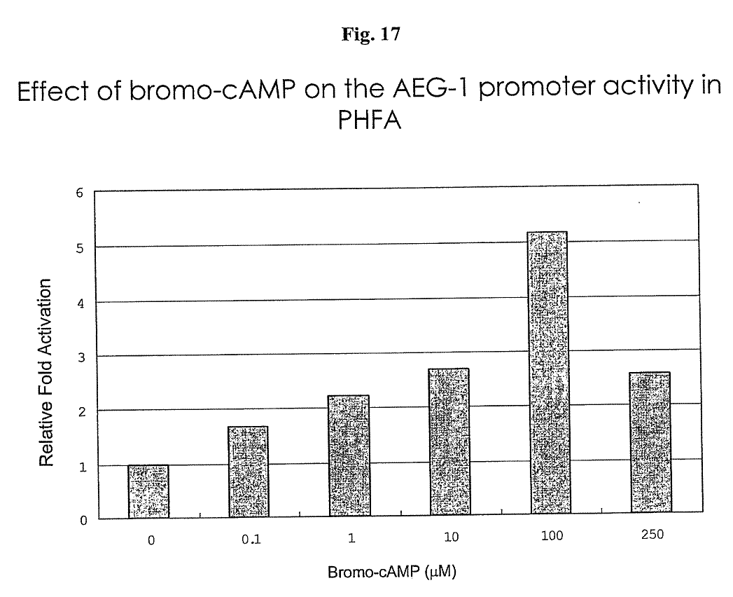

Other mediators of AEG-1 promoter activity. The HIV-Tat (Simm et al Virology 2002;294(1);1-12) and Nef (Lathi et al., Virology. 2003; 310(1):190-196) gene products were tested on the full length AEG-1 promoter at a plasmid expression vector concentration range of 50 to 800 ng/transfection point (FIG. 16). While the Tat gene product had slight (1.5-fold) stimulatory activity under the conditions tested, the Nef gene product showed approximately 5-fold activation over control at the highest amount (800 ng) of plasmid tested. Thus, the AEG-1 promoter appears to be responsive to protein products produced by HIV infection of cells. Similarly, the AEG-1 promoter showed a dose responsive stimulation of promoter activity when cells transfected with the luciferase reporter was treated with Bromo-cyclicAMP at a range of 0.1 to 100 μM (FIG. 17) when tested in primary human fetal astrocytes (PHFA).

### 8. REFERENCES

1. Sarkar, D., Kang, D.-c., Goldstein, N. I. & Fisher, P. B. (2004) Curr Genomics 5, 231-44.
2. McArthur, J. C., Haughey, N., Gartner, S., Conant, K., Pardo, C., Nath, A. & Sacktor, N. (2003) J Neurovirol 9, 205-21.
3. Minagar, A., Shapshak, P., Fujimura, R., Ownby, R., Heyes, M. & Eisdorfer, C. (2002) J Neurol Sci 202, 13-23,
4. Gorry, P. R., Ong, C., Thorpe, J., Bannwarth, S., Thompson, K. A., Gatignol, A., Vesselingh, S. L. & Purcell, D. F. (2003) Curr HIV Res 1, 463-73.
5. Bouzier-Sore, A. K., Merle, M., Magistretti, P. J. & Pellerin, L. (2002) J Physiol Paris 96, 273-82.
6. Brack-Werner, R. (1999) Aids 13, 1-22.
7. Sabri, F., Titanji, K., De Milito, A. & Chiodi, F. (2003) Brain Pathol 13, 84-94.
8. Gonzalez-Scarano, F. (1998) J Neurovirol 4, 247-8.
9. Zhang, K., McQuibban, G. A., Silva, C., Butler, G. S., Johnston, J. B., Holden, J., Clark-Lewis, I., Overall, C. M. & Power, C. (2003) Nat Neurosci 6, 1064-71.
10. Ghorpade, A., Holter, S., Borgmann, K., Persidsky, R. & Wu, L. (2003) J Neuroimmunol 141, 141-9.
11. Wang, Z., Pekarskaya, O., Bencheikh, M., Chao, W., Gelbard, H. A., Ghorpade, A., Rothstein, J. D. & Volsky, D. J. (2003) Virology 312, 60-73.
12. Doble, A. (1995) Therapie 50, 319-37.
13. Su, Z. Z., Chen, Y., Kang, D. C., Chao, W., Simm, M., Volsky, D. J. & Fisher, P. B. (2003) Gene 306, 67-78.
14. Su, Z. Z., Kang, D. C., Chen, Y., Pekarskaya, O., Chao, W., Volsky, D. J. & Fisher, P. B. (2003) J Neurovirol 9, 372-89.
15. Su, Z. Z., Kang, D. C., Chen, Y., Pekarskaya, O., Chao, W., Volsky, D. J. & Fisher, P. B. (2002) Oncogene 21, 3592-602.
16. Wang, Z., Trillo-Pazos, G., Kim, S. Y., Canki, M., Morgello, S., Sharer, L. R., Gelbard, H. A., Su, Z. Z., Kang, D. C., Brooks, A. I., Fisher, P. B. & Volsky, D. J. (2004) J Neurovirol 10 Suppl 1, 25-32.
17. Su, Z. Z., Leszczyniecka, M., Kang, D. C., Sarkar, D., Chao, W., Volsky, D. J. & Fisher, P. B. (2003) Proc Natl Acad Sci USA 100, 1955-60.
18. Kang, D. C., Gopalkcrishnan, R. V., Lin, L., Randolph, A., Valerie, K., Pestka, S. & Fisher, P. B. (2004) Oncogene 23, 1789-800.
19. Leszczyniecka, M., Kang, D. C., Sarkar, D., Su, Z. Z., Holmes, M., Valerie, K & Fisher, P. B. (2002) Proc Natl Acad Sci USA 99, 16636-41.
20. Kang, D. C., Gopalkrishnan, R. V., Wu, Q., Jankowsky, E., Pyle, A. M. & Fisher, P. B. (2002) Proc Natl Acad Sci USA 99, 637-42.
21. Gopalkrishnan, R. V., Roberts, T., Tuli, S., Kang, D., Christiansen, K. A. & Fisher, P. B. (2000) Oncogene 19, 4405-16.
22. Rich, J. N., Guo, C., McLendon, R. E., Bigner, D. D., Wang, X. F. & Counter, C. M. (2001) Cancer Res 61, 3556-60.
23. Knobbe, C. B., Merlo, A. & Reifenberger, G. (2002) Neuro-oncol 4, 196-211.
24. Frohman, M. A., Dush, M. K. & Martin, G. R. (1988) Proc Natl Acad Sci USA 85, 8998-9002.
25. Maruyama, K. & Sugano, S. (1994) Gene 138, 171-4.
26. Bahring, S., Sandig, V., Lieber, A. & Strauss, M. (1994) Biotechniques 16, 807-8.
27. Chong, S., Mersha, F. B., Comb, D. G., Scott, M. E., Landry, D., Vence, L. M., Perler, F. B., Benner, J., Kucera, R. B., Hirvonen, C. A., Pelletier, J. J., Paulus, H. & Xu, M. Q. (1997) Gene 192, 271-81.
28. Galey, D., Becker, K., Haughey, N., Kalehua, A., Taub, D., Woodward, J., Mattson, M. P. & Nath, A. (2003) J Neurovirol 9, 358-71.
29. Headley, P. M. & Grillner, S. (1990) Trends Pharmacol Sci 11, 205-11.
30. Anderson, C. M. & Swanson, R. A. (2000) Glia 32, 1-14.
31. Li, S., Mallory, M., Alford, M., Tanaka, S. & Masliah, E. (1997) J Neuropathol Exp Neurol 56, 901-11.
32. Rothstein, J. D., Van Kammen, M., Levey, A. I., Martin, L. J. & Kuncl, R. W. (1995) Ann Neurol 38, 73-84.
33. Martin, L. J., Brambrink, A. M., Lehmann, C., Portera-Cailliau, C., Koehler, R., Rothstein, J. & Traystman, R. J. (1997) Ann Neurol 42, 335-48.
34. Brown, D. M. & Ruoslahti, E. (2004) Cancer Cell 5, 365-74.
35. Sutherland, H. G., Lam, Y. W., Briers, S., Lamond, A. I. & Bickmore, W. A. (2004) Exp Cell Res 294, 94-105.
36. Ye, Z. C., Rothstein, J. D. & Sontheimer, H. (1999) J Neurosci 19, 10767-77.
37. Sontheimer, H. (2003) Trends Neurosci 26, 543-9.

Various publications are cited herein, the contents of which are hereby incorporated by reference in their entireties.

What is claimed is:

1. A method of inhibiting proliferation of a malignant cell, comprising administering, to said cell, an effective amount of an AEG-1 inhibitor which is not an anti-RAS agent.

2. The method according to claim 1, wherein the AEG-1 inhibitor is an interfering RNA that inhibits translation of AEG-1 mRNA.

3. The method of claim 1, further comprising administering an anti-RAS agent.

4. The method of claim 2, further comprising administering an anti-RAS agent.

5. The method of claim 1, wherein the malignant cell contains a mutant activated RAS gene.

6. The method of claim 1, wherein the malignant cell is selected from the group consisting of a glioblastoma multiforme cell, a breast cancer cell, a melanoma cell, a colon cancer cell, and a pancreatic cancer cell.

7. An isolated nucleic acid which is an interfering RNA that inhibits translation of an AEG-1 mRNA.

8. An isolated nucleic acid comprising an AEG-1 promoter.

9. The isolated nucleic acid of claim 8, wherein the AEG-1 promoter has a sequence as set forth in SEQ ID NO:2.

10. The isolated nucleic acid of claim 8, wherein the sequence of the AEG-1 promoter is at least 90 percent homologous to SEQ ID NO:2.

11. The isolated nucleic acid of claim 8, wherein the AEG-1 promoter is a core AEG-1 promoter comprising at least nucleic acids 1-515 of SEQ ID NO:2.

12. The core AEG-1 promoter of claim 11, wherein the sequence of the core AEG-1 promoter is at least 90 percent homologous to nucleic acids 1-515 of SEQ ID NO:2.

13. An expression construct comprising the AEG-1 promoter of claim 8, operably linked to a gene of interest.

14. The expression construct of claim 13, which is comprised in a vector.

15. The expression construct of claim 13, wherein the gene of interest is a therapeutic gene.

16. The expression construct of claim 15, wherein the therapeutic gene is an anti-cancer gene.

17. The expression construct of claim 15, wherein the therapeutic gene is an anti-glutamate gene.

17. The expression construct of claim 13, wherein the gene of interest is a reporter gene.

18. A method of inhibiting glutamate toxicity in a cell, comprising introducing, into the cell, an expression construct comprising an AEG-1 promoter operably linked to an anti-glutamate gene under conditions such that the anti-glutamate gene is expressed.

19. The method of claim 18, wherein the anti-glutamate gene is an EAAT2 gene.

20. A method of treating a viral infection in a subject, comprising introducing, into a cell of the subject, an expression construct comprising an AEG-1 promoter operably linked to an anti-viral gene under conditions such that the anti-viral gene is expressed.

21. The method of claim 20, wherein the viral infection is HIV-1 infection.

22. The method of claim 21, wherein the anti-viral gene is an interferon alpha gene.

23. A method of treating HAD in a subject, comprising introducing, into a cell of the subject, an expression construct comprising an AEG-1 promoter operably linked to a therapeutic gene selected from the group consisting of a gene that augments immunity, an anti-viral gene, and an anti-glutamate gene, under conditions such that the therapeutic gene is expressed.

24. A method of augmenting immunity in a subject, comprising introducing, into a cell of the subject, an expression construct comprising an AEG-1 promoter operably linked to a therapeutic gene selected from the group consisting of an interferon alpha gene, an interferon beta gene, an interferon gamma gene, an interleukin 2 gene, an interleukin 4 gene, and an interleukin 12 gene, under conditions such that the therapeutic gene is expressed.

25. An assay for identifying an inhibitor of AEG-1, comprising:
(i) exposing a cell containing an expression construct comprising an AEG-1 promoter operably linked to a reporter gene to a test agent;
(ii) measuring the amount of expression of the reporter gene;
(iii) comparing the amount of expression of the reporter gene measured in step (ii) to the amount of reporter gene expression in a control cell not exposed to the test agent;
wherein a test agent that decreases the expression of the reporter gene relative to control values is an AEG-1 inhibitor.

26. An assay for identifying an inhibitor of AEG-1, comprising:
(i) exposing a cell containing an expression construct comprising an EAAT2 promoter operably linked to a reporter gene to a test agent;
(ii) measuring the amount of expression of the reporter gene;
(iii) comparing the amount of expression of the reporter gene measured in step (ii) to the amount of reporter gene expression in a control cell not exposed to the test agent;
wherein a test agent that increases the expression of the reporter gene relative to control values is an AEG-1 inhibitor.
